(19) United States
(12) Patent Application Publication
(10) Pub. No.: US 2026/0102540 A1
(43) Pub. Date: Apr. 16, 2026

(54) NANOFIBER-HYDROGEL COMPOSITES AND METHODS FOR INHIBITING ADHESION FORMATION

(71) Applicant: The Johns Hopkins University, Baltimore, MD (US)

(72) Inventors: Hai-Quan Mao, Baltimore, MD (US); Sami Tuffaha, Baltimore, MD (US); Sashank Reddy, Baltimore, MD (US); Visakha Suresh, Baltimore, MD (US)

(21) Appl. No.: 19/116,866

(22) PCT Filed: Sep. 30, 2023
(86) PCT No.: PCT/US2023/075657

§ 371 Date: Mar. 28, 2025

Related U.S. Application Data
(60) Provisional application No. 63/412,062, filed on Sep. 30, 2022.

(51) Int. Cl. A61L 27/48; A61L 27/18; A61L 27/20; A61L 27/24; A61L 27/36; A61L 27/52; A61L 27/54

(52) U.S. Cl. CPC A61L 27/48; A61L 27/18; A61L 27/20; A61L 27/24; A61L 27/3675; A61L 27/52; A61L 27/54; A61L 2300/404; A61L 2300/41; A61L 2430/32

(57) ABSTRACT

Please insert the following Abstract on a separate sheet at the end of the application: Provided herein, inter alia, are hydrogel composite, its compositions, and use thereof for treatment of undesired adhesion formation and treatment of peripheral nerves, e.g., by preventing scarring and adhesion of peripheral nerves.

### CROSS-REFERENCES TO RELATED APPLICATIONS

This application claims the benefit of priority of U.S. Provisional Application No. 63/412,062 filed on Sep. 30, 2022, which is incorporated herein by reference in its entirety and for all purposes.

### TECHNICAL FIELD

Provided herein, inter alia, are hydrogel composites, its compositions, and use for treatment or reduction of peripheral nerves, e.g., by preventing scarring and adhesion of peripheral nerves.

### BACKGROUND

Nerve scarring and adhesions represent a major problem lacking an acceptable solution. Adhesions are a type of scar tissue which may connect organs and tissues that are not typically connected. They may form as a result from multiple types of trauma or inflammation, such as surgery, wounding, infection, radiation or other disease.

Following nerve decompression and neurolysis, scar formation and adhesion to the wound bed can lead to recurrent symptoms. Surgery alone is used to address this problem leading to poor outcomes.

It would be desirable to have new treatments to address scarring and adhesion.

### SUMMARY

In one aspect, we now provide fiber-hydrogel or nanofiber-hydrogel composites (“hydrogel composites” or “hydrogel composite” or “hydrogel composition”) for treating, preventing or suppressing adhesion formation or proliferation and/or scarring.

In one aspect, nanofiber-hydrogel composites or compositions are used for treating, preventing or suppressing perineural adhesion and/or scarring in a subject.

In an aspect, provided is a method of treating neural tissues in a subject. In one embodiment, the method includes applying a hydrogel composition to or around the neural tissues (e.g., peripheral nerves).

In a further aspect, methods are provided using the present nanofiber-hydrogel composites or compositions for treating, preventing or suppressing formation of post surgical adhesions. The surgical procedure includes, among others, surgery for treatment of carpal tunnel syndrome, knee surgery, ankle surgery, shoulder surgery, elbow surgery, or other joint surgery.

In an additional aspect, methods are methods are provided using the present nanofiber-hydrogel composites or compositions for treating or preventing injury or discomfort involving a subject's tendon. Such methods may include surgical procedures for treating a subject suffering from carpal tunnel syndrome.

In a further aspect, methods are provided for treating or preventing or reducing the occurrence of arthrofibrosis, including knee arthrofibrosis. The methods in general comprise administering a hydrogel composition to a subject in need thereof (e.g. a patient that is undergoing a knee or other joint surgery, or suffered from knee or other joint trauma or injury).

In an additional aspect, methods are provided for treating a subject suffering from carpal tunnel syndrome, which includes administering a hydrogel composition (also referred to as nanofiber-hydrogel composites or compositions) as disclosed herein to the subject.

In embodiments, the subject may have undergone surgical treatment for carpal tunnel syndrome and the hydrogel composition is administered to the surgically treated subject.

In additional embodiments, the subject may be first treated hydrogel composition (also referred to as nanofiber-hydrogel composites or compositions) as disclosed herein and thereafter undergo a surgical procedure such as a procedure to treat one or more tendons, or for treatment of carpal tunnel syndrome.

In additional embodiments, the subject may have experienced one or more symptoms of carpal tunnel syndrome (e.g. hand, finger or wrist pain) and the hydrogel composition (also referred to as nanofiber-hydrogel composites or compositions) is administered to the subject exhibiting such symptoms. In such embodiments, in one aspect, the subject may not have undergone surgery for carpal tunnel syndrome. Alternatively, in such embodiments, in one aspect, the subject may have previously undergone surgery for carpal tunnel syndrome.

The methods in general comprise administering a hydrogel composition (also referred to as nanofiber-hydrogel composites or compositions) to a subject in need thereof (e.g. a patient that is undergoing a procedure involving a patient's tendons such as to treatment carpal tunnel syndrome, or knee or other joint surgery, or a patient that has suffered from knee or other joint trauma or injury).

In a yet further aspect, methods are provided for treating or preventing or reducing the occurrence of adhesions that may occur through radiation-induced fibrosis in a subject, including radiation-induced fibrosis in the lung.

The present methods include treating or minimizing reducing fibrotic lesions following radiotherapy in tissue, including skin, lung and liver. The methods in general comprise administering an effective amount of a hydrogel composition (also referred to as nanofiber-hydrogel composites or compositions) to a subject in need thereof (e.g. a patient that is undergoing radiotherapy. e.g. to treat a cancer). The administration suitably may be localized to a targeted site, such as by injection.

In a still further aspect, methods are provided for treating radiation-induced fibrosis in a subject, including radiation-induced fibrosis in the lung.

In certain aspects, the treatment methods may include administering or other placing of a hydrogel composition (also referred to as nanofiber-hydrogel composites or compositions) at the interface between healing tissues and the surrounding tissues.

In embodiments, the hydrogel composition (also referred to as nanofiber-hydrogel composites or compositions) is injected or implanted on or around the neural tissues, or alternatively, is for dermal or subdermal administration into the neural tissues of the subject.

In certain aspects, the subject is identified and selected for treatment based on a condition disclosed herein, and a hydrogel composition (also referred to as nanofiber-hydrogel composites or compositions) as disclosed herein is administered to the identified and selected subject.

For instance, the subject may be identified and selected as being in need of treatment for perineural adhesion and/or scarring, and a hydrogel composition as disclosed herein is administered to the identified and selected subject.

The subject also may be identified and selected for treatment (including preventing or suppressing) of formation of post-surgical adhesions, and a hydrogel composition as disclosed herein is administered to the identified and selected subject.

The subject also may be identified and selected for treatment of radiation-induced fibrosis, and a hydrogel composition as disclosed herein is administered to the identified and selected subject.

The subject also may be identified and selected for treatment of carpal tunnel syndrome, and a hydrogel composition as disclosed herein is administered to the identified and selected subject.

In certain aspects, the subject may not be suffering form or otherwise in need of treatment of wound healing with a hydrogel composition as disclosed herein. In certain aspects, the subject may not be suffering form or otherwise in need of a dermal filler in a treatment area where hydrogel composition as disclosed herein is administered.

In particular aspects, the present hydrogel composition includes a functionalized hyaluronic acid network and an associated fiber or scaffold component. In preferred aspects, the nanofiber-hydrogel composition is capable of at least one of i) suppressing scarring, ii) suppressing adhesion of the neural tissues, and/or iii) promoting regeneration of the neural tissue in the subject.

The hydrogel composition suitably comprises 1) a fiber or scaffold component; 2) hyaluronic acid including functionalized hyaluronic acid; and preferably 3) a crosslinking component.

Suitably the fiber component comprises one or more polymer materials, and/or one or more extracellular matrix materials.

In a preferred aspect, the composition comprises substantially non-spherical microbeads comprising a functionalized hyaluronic acid network which may be covalently linked to a fiber of scaffold component.

In an embodiment, the fiber component comprises non-woven polymeric fiber. In certain embodiments, the polymeric fiber includes an electrospun polycaprolactone fiber. Optionally, the polymeric fiber includes a synthetic polymeric material comprising for example a poly(lactic-co-glycolic acid), a polylactic acid, and/or a polycaprolactone, or a combination thereof. In one embodiment, the complex is formulated to be substantially biocompatible. Optionally, the polymeric fiber includes a biological polymeric material that includes a silk, a collagen, a chitosan, and/or a combination thereof.

In an embodiment, the fiber component comprises a collagen material.

In one embodiment, the hydrogel material includes hyaluronic acid. Optionally, the hydrogel material includes a hydrogel material that includes a poly (ethylene glycol), a collagen, a dextran, an elastin, an alginate, a fibrin, a alginate, a hyaluronic acid, a poly(vinyl alcohol), a derivative thereof, or a combination thereof.

In one embodiment, the fiber of scaffold component may comprise polycaprolactone or other polymer fibers having a mean length of less than about 200 micrometers.

In one embodiment, in a composite that comprises non-spherical microbeads, the composite may comprise a crosslinking agent for example present at a concentration from about 1 mg/mL to about 25 mg/mL, preferably wherein the mean size of the microbeads is within the range of about 50 micrometers to about 300 micrometers along the longest dimension, suitably wherein the microbeads are pre-reacted (e.g. the crosslinking agent has reacted with one or more other components on the microbeads), and/or preferably wherein the microbeads are substantially stable at room temperature for at least about 6 months.

In certain embodiments, a composite composition may be formed combining hyaluronic acid (including functionalized hyaluronic acid), the fiber or scaffold component (e.g. polycaprolactone nanofibers or a collagen materials), and a crosslinking agent. In certain embodiments, the functionalized hyaluronic acid includes thiolated hyaluronic acid, and the crosslinking agent includes poly(ethylene glycol) diacrylate (PEGDA), or a derivative thereof. In one aspect, if used, a plurality of polycaprolactone fibers may be formed by electrospinning. The plurality of polycaprolactone fibers may include an electrospun fiber.

In embodiments, the diameter of the nanofibers such as polycaprolactone nanofibers is within the range from 10 to 200 nanometers, or 20 to 100 or 150 nanometers.

In embodiments, a weight ratio between the functionalized hyaluronic acid and the nanofibers suitably ranges from 1 to 10 or 10 to 1; or 2 to 8 or 8 to 2; or 3 to 7 or 7 to 3; or 4 to 6 or 6 to 4.

In some embodiments, the hydrogel composition (also referred to as nanofiber-hydrogel composites or compositions) further includes a compound selected from the group consisting of growth factors, compounds stimulating angiogenesis, immunomodulators, inhibitors of inflammation, and combinations thereof. In some embodiments, the hydrogel composite further includes one or more compounds that have therapeutic effects, vascularization effects, anti-vascularization effects, anti-inflammatory effects, anti-bacterial effects, antihistamine effects, and combinations thereof.

In particular embodiments, the hydrogel composition (also referred to as nanofiber-hydrogel composites or compositions) includes a Botulinum neurotoxin (BoNT) particularly a Botulinum toxin type A (BoNTA) material (e.g. Botox or Dysport).

In an aspect, provided is an implant for treating a subject after neurosurgery. The implant includes the hydrogel composite (also referred to as nanofiber-hydrogel composites or compositions) as described herein. The implant is suitably applied on or around neural tissues of the subject.

Further provided is a kit including the implant as described herein and an applicator. In some embodiments, the applicator is an injection syringe.

In some embodiments, the implant may be dehydrated. The kit may further include a vial containing water, saline solution or suitable fluid for reconstitution of the dehydrated implant.

The terms hydrogel composite or composition, nanofiber-hydrogel composites or compositions, and nanofiber/hyaluronic acid hydrogel composite (NHC) are used interchangeably herein.

Where applicable or not specifically disclaimed, any one of the embodiments described herein are contemplated to be able to combine with any other one or more embodiments, even though the embodiments are described under different aspects of the invention.

Other aspects are disclosed infra.

### BRIEF DESCRIPTION OF THE DRAWINGS

FIG. 1A schematically shows engineering a nanofiber-hydrogel composite (NHC) based on interfacial bonding between thiolated hyaluronic acid (HA-SH) and PCL materials.

FIG. 1B-1C show scanning electron microscopy images of rat native fat tissue (FIG. 1B) and PCL nanofiber-HA hydrogel composite (FIG. 1C). Fibers are embedded into the HA hydrogel network (arrowheads) that mimics fat tissue matrices.

FIG. 1D shows images of a PCL nanofiber-HA hydro-gel composite (oscillatory shear storage modulus G′=250 Pa, left) and an HA hydrogel (G′0=80 Pa, right) construct-ed from the same 80-Pa HA hydrogel.

FIG. 1E shows an image of the composite that can be injected through a 30-gauge needle.

FIG. 1F shows a scheme to formulate a hydrogel composite according an exemplary embodiment of the disclosure.

FIG. 1G shows a scheme to modify polycarprolactam nanofibers as described in Example 1 according an exemplary embodiment of the disclosure.

FIGS. 2A-2B show histological analysis of the sciatic nerve-NHC interface with hematoxylin and eosin (H&E) staining for the control group (FIG. 2A) and experimental group (FIG. 2B). The nerve aligned well with the NHC as shown in FIG. 2B. Scale bar: 200 um.

FIGS. 2C-2D show hematoxylin and eosin (H&E) staining for the control group (FIG. 2C) and experimental group (FIG. 2D). The nerve aligned well with the NHC to present a smooth interface as compared to control group. Scale bar: 50 um.

FIGS. 2E-2F show Masson's Trichrome (MT) staining for the control group (FIG. 2E) and experimental group (FIG. 2F). Minimal collagen deposition (stained blue) was seen in the experimental group compared to control, indicating a decrease in scar formation in the animals treated with the NHC. Scale bar: 1 mm.

FIG. 3 (includes FIGS. 3A-3J) shows storage moduli (G′) of samples of Example 3.

FIG. 4 shows a procedure with a rtest subject, as discussed in Example 4.

FIG. 5 (includes FIGS. 5A-5B) and FIG. 6 shows results of Example 4 which follows.

FIG. 7 (includes FIGS. 7A-7B) and FIG. 8 (includes FIGS. 8A-8B) shows results of Example 4 which follows, including that animals treated with the NHC (hydrogel composite) had a substantial up-regulation of anti-inflammatory cytokines TGF-β1 and IL-10.

### DETAILED DESCRIPTION

The following detailed description, given by way of example, but not intended to limit the invention solely to the specific embodiments described, may best be understood in conjunction with the accompanying drawings.

In one aspect, a pre-reacted, beaded composite material is provided that comprises a hydrogel and a nanostructure for use in methods for reducing or avoiding formation or proliferation of adhesions and/or scar tissue. We also provide a device comprising beaded composite materials for cell and tissue delivery for or avoiding formation or proliferation of adhesions and/or scar tissue.

The invention also relates to composite materials that can recruit, capture, encapsulate, associate, and/or embed specific tissue constituents including but not limited to adipocytes, other mesenchymal cells, or mesenchymal stem cells. The invention further relates to composite materials that can recruit, capture, encapsulate, associate, and/or embed specific tissues including but not limited to adipose tissues.

In particular aspects, it has been found that the composite materials as disclosed herein can promote cellular infiltration, polarization of macrophages away from M1 phenotype (associated with fibrosis and scarring) to M2 phenotype (associated with angiogenesis and regeneration), and eventual replacement by host tissues (13, 14). Additionally, preferred composite materials can be readily y degraded via enzymatic hydrolysis that further allows for local extracellular matrix remodeling and angiogenesis, as macrophages and other host cells secrete hyaluronidase, matrix metalloproteases (MMPs), and cytokines such as vascular endothelial growth factor (VEGF). These fundamental characteristics make the composite materials effective for the prevention or minimizing of perineural adhesions.

As further discussed in the examples below, in our analyses, rats underwent bilateral circumferential sciatic nerve neurolysis and mechanical irritation of the surrounding wound bed. Primary neurolysis cohorts underwent neurolysis and mechanical irritation then were either treated with the composite material (NHC) perineurally, or left untreated. Secondary neurolysis cohorts were all untreated after initial neurolysis and mechanical irritation. 8 weeks later repeat neurolysis and mechanical irritation, then animals similarly treated with NHC or untreated. Endpoint analyses was similarly performed 8 weeks following secondary re-exposure. Endpoint analysis was performed 8 weeks later using biomechanical testing to assess the breaking point of the perineural adhesions surrounding the sciatic nerve, perineural collagen deposition and expression of inflammatory cytokines. Results: In NHC-treated animals, the average force required to dislodge the sciatic nerve from its wound bed was 0.69±0.11 and 2.02±0.43 Newtons, in primary and secondary neurolysis cohorts, respectively. In untreated controls, the nerve could not be dislodged; the average maximum force before nerve rupture was 2.98±0.57 and 2.77±0.18 Newtons, in primary and secondary neurolysis cohorts, respectively. In the primary neurolysis cohort, average percentage of perineural collagen deposition was 21.4±4.4 and 4.6±1.4% in control and experimental groups, respectively (p<0.0001). In the secondary neurolysis cohort, this was 47.8±5.8 and 16.7±3.7% in control and experimental groups, respectively (p<0.0001). Conclusion: Treatment with NHC at the time of primary or secondary surgical exposure and neurolysis reduces perineural adhesion formation, decreases collagen deposition, and increases upregulation of anti-inflammatory gene expression. The NHC thus is shown to be effective to prevent or minimize perineural adhesion formation in patients with with peripheral nerve injuries or compressive neuropathy.

In particular aspects, the administered materials is a fiber-hydrogel composite comprising: a) fibers comprising one or more extracellular matrix proteins (ECM); b) a hyaluronic acid (HA); and c) a crosslinking agent, suitably wherein the fibers are nanofibers or microfibers, and suitably wherein the HA is bonded to the fibers by the crosslinking agent to form a composite network.

Suitably, in preferred fiber-hydrogel composites, the fibers comprise one or more selected from collagen, gelatin, cellulose, modified cellulose, cellulose acetate, HPMC, ethyl cellulose, silk, chitosan, keratin, elastin, elastin-like polypeptides, tropoelastin, and hyaluronic acid.

In certain preferred fiber-hydrogel composites, the fibers comprise one or more from bovine type I collagen or gelatin, and its derivatives.

In certain preferred fiber-hydrogel composites, the one or more ECMs comprise a collagen nanofiber.

Certain preferred fiber-hydrogel composites comprise a collagen nanofiber that comprises a type I bovine collagen nanofiber or fragments thereof.

Certain preferred fiber-hydrogel composites comprise a collagen nanofiber is electrospun or centrifugal spun.

Certain preferred fiber-hydrogel composites comprise HA is covalently bonded to the type I bovine collagen nanofiber sheet or fragments thereof.

Certain preferred fiber-hydrogel composites comprise polycaprolactone fiber.

Certain preferred fiber-hydrogel composites comprise a crosslinking agent that generates interfacial bonding between the collagen nanofiber and the HA chain.

In certain preferred fiber-hydrogel composites, a collagen nanofiber is retained inside the fiber-hydrogel composite.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. In certain embodiments, the subject is a human patient or an animal subjected to medical treatment.

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The hydrogel may contain “water-swellable” polymer is one that absorbs an amount of water greater than at least 50% of its own weight, upon immersion in an aqueous medium. The polymeric matrix may be formed of any suitable synthetic or naturally occurring polymer material. As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

In certain embodiments, the hydrogel is a composite or composite material. The term “composite” as used herein includes any association, bonding or attachments of two or more components. In some embodiments, the “hydrogel composite” as used herein include at least a polymeric fiber and a hydrogel material. The hydrogel composite contains the polymeric fiber (e.g., polycaprolactone) and hydrogel material (e.g., hyaluronic acid (HA)).

A term “functional network” as used herein means that the interactions between components results in a chemical, biochemical, biophysical, physical, or physiological benefit. In addition, a functional network may include additional components, including cells, biological materials (e.g., polypeptides, nucleic acids, lipids, carbohydrates), therapeutic compounds, synthetic molecules, and the like. In certain embodiments, the scaffold complex promotes tissue growth and cell infiltration when implanted into a target tissue present in a human subject.

The term “nanofiber” as used herein refers to a fibrous material having at least one dimension (e.g., length, or width) less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a length less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a width less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm.

The term “nanofiber-hydrogel composite” as used herein refers to a composite including at least nanofibers (e.g., polymeric fibers) and hydrogel (e.g., HA), which form functional networks. In addition, the “nanofiber-hydrogel composite,” “hydrogel composite,” “composite” or “complex” as used herein are interchangeably used referring to such composite including at least nanofibers (e.g., polymeric fibers) and hydrogel (e.g., HA).

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da. Polymers and oligomers may be naturally occurring or obtained from synthetic sources.

As used herein, the term “biomaterial” means an organic material that has been engineered to interact with biological systems. In some embodiments of the invention, a biomaterial is a hydrogel. In some embodiments, biomaterial is a bacterially derived hyaluronic acid (HA).

As used herein, the term “biodegradable” refers to a material that can be broken down by biological means in a subject.

As used herein, the term “implantable” means able to be formulated for implantation via a syringe to a subject.

As used herein, the term “soft tissue” refers to tissues that connect, support, or surround other structures and organs of the body. Soft tissue includes muscles, tendons, ligaments, fascia, nerves, fibrous tissues, fat, blood vessels, and synovial membranes.

As used herein, the term “stable” refers to a material that does not degrade at room temperature.

As used herein, the term “functionalized” refers to a material that is uniformly or non-uniformly modified so as to have a functional chemical moiety associated therewith (e.g., chemically modified). In some cases, functional chemical moiety is capable of reacting to permit the formation of a covalent or non-covalent bond. In some cases, functional chemical moiety can provide the material improved properties.

### Nanofiber-Hydrogel Composite

Provided is a “nanofiber-hydrogel composite or composition,” “hydrogel composite,” or “hydrogel composite” that is formed by combining hydrogel materials or other biomaterials with polymeric nanofibers. The composite may be formulated such that the density, ratio of gel to fibers, and other properties are variable, while maintaining sufficient porosity and strength.

A ratio of polymeric nanofibers to hydrogel material can be determined my any means known in the art. For example, the ratio of polymeric fiber to hydrogel material is from about 1:100 to about 100:1 on a component-mass basis, such as about 1:50 to about 50:1, or 1:10 to about 10:1, such as 1:5 to about 5:1, such as about 1:3 to about 3:1. The ratio of polymeric fiber to hydrogel material is also provided as a concentration basis, e.g., a given weight of polymeric fiber per volume of hydrogel material. For example, the concentration is from about 1 to 50 mg/mL. The hydrogel material is generally disposed on the polymer fiber, such as being bonded to the outer surface (or an outer surface, depending upon the composition and shape) of the polymer fiber. The scaffold complex is not generally a uniform solid material.

The composite may contain a plurality of pores present on or within a surface of the composite. The presence, size, distribution, frequency and other parameters of the pores can be modulated during the creation of the composite, hydrogel, or nanofibers. Pore size can be from below about 1 nm to up to 100 nm, including 1, 2, 3, 45, 10, 15, 20, 30, 40, 50, 6070, 80, 90 or 100 nm, and the size thereof may be narrowly tailored, e.g., such that at least 40%, such as 50%, 60%. 70%, 80%. 90%, 95% or greater than 95% of the pores are in a desired size or within a desired size range.

The composite may be suitable for incorporation into a tissue of a human subject, and thus they are generally “biocompatible”, meaning capable of interacting with a biological system (such as found in a human subject) without inducing a pathophysiological response therein and/or thereby. In some embodiments, the composite is provided in order to be durably retained in the tissue, e.g., nerve tissues. Alternatively, the composite may be transiently retained in the human subject and are provided as substantially biodegradable. Preferably, the polymeric fibers or nanofibers in the composite include biocompatible biodegradable polymers, e.g., biocompatible biodegradable polyester. In certain embodiments, the polymeric fibers or nanofibers include polycaprolactone. In certain embodiments, the polymeric fibers or nanofibers are polycaprolactone.

To achieve fiber-reinforcement effect while maintaining high porosity in the hydrogel phase, an electrospun fiber-hydrogel composite that offers superior properties as compared to other complex is provided. Such a composite design not only allows stronger mechanical reinforcement from the solid fiber component, but also allows independent tuning of bulk mechanical properties and the average pore size/porosity of the hydrogel phase, enabling both optimal cell infiltration properties and structural integrity.

To further achieve the desired effects, in some embodiments, a crosslinking agent is preferably used to introduce crosslinking between the nanofibers and also between the nanofibers and the hydrogel. For instance, suitable crosslinking agents include for example materials with one or preferably two or more reactive groups such as hydroxy, carboxy, thio, or amino. Preferred crosslinking agents include glycol compounds such as a poly(ethylene glycol), including poly(ethylene glycol), thiolated poly(ethylene glycol), and/or poly(ethylene glycol) diacrylate (PEGDA). Use of a crosslinking agent can for example help extend durability of the product, and allows for modulation of crosslinking density in order to achieve optimal other properties.

### Gel/Hydrogel Component

In an aspect, the composite includes a hydrogel having three-dimensional network of polymers (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. In some embodiments, the hydrogel may be biodegradable. The hydrogel can include any type of suitable hydrogel component known in the art. The gel and/or hydrogels can be formed of any suitable synthetic or naturally-occurring materials.

In some embodiments, hydrogel materials are functionalized. In particular embodiments, hydrogel materials are functionalized with groups comprising hydroxyl, amino, carboxyl, thio, acrylate, sulfonate, phosphate, amide, as well as modified forms thereof, such as activated or protected forms.

The hyaluronic acid (HA) is preferably used as the hydrogel material. HA is a non-sulfated, linear polysaccharide with repeating disaccharide units which form the hydrogel component. HA is also a non-immunogenic, native component of the extracellular matrix in human tissues, and widely used as a dermal filler in aesthetic and reconstructive procedures.

Breakdown of HA is facilitated by native hyaluronidases whose expression is increased in areas of tissue damage and inflammation. Importantly, studies have shown that small HA degradation fragments of 3-10 disaccharide units are potent regulators of endothelial cell proliferation, migration, tubule formation, and angiogenesis. These biological functions of HA are thought to be mediated via CD44 in a pathway involving Ras and PKC. Blockade of CD44/HA interactions using anti-CD44 antibodies reduced proliferation and migration of human microvascular endothelial cells in vitro. HA hydrogels have been investigated as potential matrices for cell delivery in a variety of models of cell and tissue injury. These hydrogels can serve as a protective and supporting scaffold for cells and can also reduce scarring. Thus, it is believed HA has a critical role in enhancing tissue regeneration by promoting cell infiltration and promoting angiogenesis.

The molecular weight of hyaluronic acid may affect the overall properties of the composite. In some embodiments, the molecular wight of HA (e.g., HA-SH) may be at least about or greater than 10 kDa, at least about or greater than 50 kDa, at least about or greater than 100 kDa, at least about or greater than 200 kDa, at least about or greater than 300 kDa, at least about or greater than 400 kDa, at least about or greater than 500 kDa, at least about or greater than 600 kDa, at least about or greater than 700 kDa, at least about or greater than 800 kDa, at least about or greater than 900 kDa, at least about or greater than 1.0 MDa, at least about or greater than 1.5 MDa, at least about or greater than 2.0 MDa, at least about or greater than 2.5 MDa, at least about or greater than 3.0 MDa.

In some embodiments, hyaluronic acid are functionalized. In particular embodiments, hyaluronic acid are functionalized with groups comprising hydroxyl, amino, carboxyl, thio, acrylate, sulfonate, phosphate, amide, as well as modified forms thereof, such as activated or protected forms. In certain embodiments, the hydrogel material includes a hyaluronic acid (HA). In certain embodiments, the hydrogel material includes functionalized hyaluronic acid (HA). In other preferred embodiments, the hydrogel material includes acrylated hyaluronic acid (HA). In some embodiments, the hydrogel material includes thiolated hyaluronic acid (HA).

In some embodiment, the HA of the invention is a sterilized HA, e.g., chemically and/or physically sterilized or derived from bacterial fermentation.

Further, the polymer component of the hydrogels may also include a cellulose ester, for example, cellulose acetate, cellulose acetate propionate (CAP), cellulose acetate butyrate (CAB), cellulose propionate (CP), cellulose butyrate (CB), cellulose propionate butyrate (CPB), cellulose diacetate (CDA), cellulose triacetate (CTA), or the like. In some embodiments, the gels/hydrogels may include other water-swellable polymers, such as acrylate polymers, which are generally formed from acrylic acid, methacrylic acid, methyl acrylate, ethyl acrylate, methyl methacrylate, ethyl methacrylate, and/or other vinyl monomers.

### Nanofibers

In an aspect, the composite also includes polymeric fibers, generally having a mean diameter of from about 10 nm to about 10,000 nm, such as about 100 nm to about 8000 nm, or about 150 nm to about 5,000 nm, or about 100, 150, 200, 250, 300, 350, 400, 450, 500, 600, 700, 800, 900, 1,000, 1,500, 2,000, 2,500, 3,000, 3,500, 4,000, 4,500, 5,000, 5,500, 6,000, 6,500, 7,000, 7,500, or 8,000. The polymeric fiber generally has a mean length of from about 10 μm to about 500 μm, such as about 10, 50, 100, 150, 200, 250, 300, 350, 400, 450, or 500 μm.

In certain embodiments, the polymeric fibers are nanofibers generally having a mean diameter of less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the polymeric fibers are nanofibers generally having a length less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. The length of the nanofibers is determined using optical fluorescence microscopy or electron microscopy.

In some embodiments, nanofibers are functionalized. In some embodiments, fibers are functionalized with groups comprising hydroxyl, amino, carboxyl, thio, acrylate, sulfonate, phosphate, maleimide, amide, as well as modified forms thereof, such as activated or protected forms.

Preferably, the polymeric fibers or nanofibers in the composite include biocompatible biodegradable polymers, e.g., biocompatible biodegradable polyester. In certain embodiments, the polymeric fibers or nanofibers include polycaprolactone. In certain embodiments, the polymeric fibers or nanofibers are polycaprolactone.

The nanofibers may include, but not limited to, nanofibers, nanotubes, nanofilaments, mesh sections, branched filaments or networks. The nanofibers may also comprise any suitable chemical functional groups to facilitate the covalent or noncovalent crosslinking between the nanofibers and the polymers of the hydrogels of the invention. Method, techniques, and materials are well known in the art for making and functionalizing nanofibers.

In certain embodiments, microfabrication methods are used to make the nanofibers. In various embodiments, the disclosed devices can be assembled and/or manufactured using any suitable microfabrication technique. Such methods and techniques are widely known in the art.

The nanofibers may also be fabricated by electrostatic spinning (also referred to as electrospinning). The process of electrospinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening).

Electrostatically spun fibers can be produced having very thin diameters. Parameters that influence the diameter, consistency, and uniformity of the electrospun fibers include the polymeric material and cross-linker concentration (loading) in the fiber-forming combination, the applied voltage, and needle collector distance.

Preferably, the nanofiber has a diameter ranging from about 1 nm to about 100 mm. In some embodiments, the nanofiber has a diameter in a range of about 1 nm to about 1000 nm. Further, the nanofiber may have an aspect ratio in a range of at least about 10 to about at least 100. It will be appreciated that, because of the very small diameter of the fibers, the fibers have a high surface area per unit of mass. This high surface area to mass ratio permits fiber-forming solutions or liquids to be transformed from liquid or solvated fiber-forming materials to solid nanofibers in fractions of a second.

### Crosslinking

The preferred form of interaction of the complex/comp containing polymer fibers and hydrogel includes a crosslinking moiety, generally present in an amount effective to introduce bonding between polymer fiber and hydrogel material, e.g., to induce crosslinking between polycaprolactone fiber and hyaluronic acid.

For certain applications, particularly when high cohesive strength is desired, the polymers of the gel/hydrogels of the invention may be covalently crosslinked. The disclosure contemplates that crosslinking may be desired as between the polymers of the gel/hydrogel component, but also crosslinking may be desired as between the polymers of the gel/hydrogel and the nanostructure components of the composite materials of the invention. The invention contemplates any suitable means for crosslinking polymers to one another, and crosslinking the gel/hydrogel polymers with the nanostructure components of the invention. The gel/hydrogel polymers may be covalently crosslinked to other polymers or to the nanostructures, either intramolecularly or intermolecularly or through covalent bonds. In the former case, there are no covalent bonds linking the polymers to one another or to the nanostructures, while in the latter case, there are covalent crosslinks binding the polymers to one another or to the nanostructures. The crosslinks may be formed using any suitable means, including using heat, radiation, or a chemical curing (crosslinking) agent. The degree of crosslinking should be sufficient to eliminate or at least minimize cold flow under compression. Crosslinking also includes the use of a third molecule, a “cross-linker” utilized in the cross-linking process.

“Cross-linkers” or “Cross-linking agents” may be suitably chosen, for example, from the group of poly(ethylene glycol) PEG, e.g. thiolated poly(ethylene glycol), poly(ethylene glycol) diacrylate (PEGDA), or derivatives thereof.

For thermal crosslinking, a free radical polymerization initiator is used, and can be any of the known free radical-generating initiators conventionally used in vinyl polymerization. Preferred initiators are organic peroxides and azo compounds, generally used in an amount from about 0.01 wt. % to 15 wt. %, preferably 0.05 wt. % to 10 wt. %, more preferably from about 0.1 wt. % to about 5% and most preferably from about 0.5 wt. % to about 4 wt. % of the polymerizable material. Suitable organic peroxides include dialkyl peroxides such as t-butyl peroxide and 2,2bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds include azo bis (isobutyronitrile) and azo bis (2,4-dimethylvaleronitrile). The temperature for thermally crosslinking will depend on the actual components and may be readily deduced by one of ordinary skill in the art, but typically ranges from about 80° C. to about 200° C.

Crosslinking may also be accomplished with radiation, typically in the presence of a photoinitiator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG. Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa(3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri(3-mercaptopropionate), trimethylolethane trithioglycolate, trimethylolpropane tri(3-mercaptopropionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

The polymers and/or nanostructures may also be crosslinked prior to admixture with the complementary oligomer. In such a case, it may be preferred to synthesize the polymer in crosslinked form, by admixing a monomeric precursor to the polymer with multifunctional comonomer and copolymerizing. Polymerization may be carried out in bulk, in suspension, in solution, or in an emulsion. Solution polymerization is preferred, and polar organic solvents such as ethyl acetate and lower alkanols (e.g., ethanol, isopropyl alcohol, etc.) are particularly preferred.

In some embodiments, is a chemical crosslinking agent is employed, the amount used will preferably be such that the weight ratio of crosslinking agent to hydrophilic polymer is in the range of about 1:100 to 1:5. To achieve a higher crosslink density, if desired, chemical crosslinking suitably may be combined with radiation curing.

In some embodiments, as discussed above the crosslinking agent includes a poly(ethylene glycol) such as for example poly(ethylene glycol) diacrylate (PEGDA). In certain embodiments, when the functionalized hyaluronic acid comprises thiolated hyaluronic acid, the crosslinking agent includes poly(ethylene glycol) diacrylate (PEGDA).

### Microbeads

As discussed, in various aspects, it can be preferred that the composites/hydrogels are formed into particulate formulations, enabling use of higher concentrations of each component and enhanced stability. In a preferred aspect, a system of particle may be employed wherein the pre-formed hydrogel-nanofiber composite is physically modulated, such as by being pushed through one, two, three, or more than three mesh screens, creating a population of nonspherical beads that are relatively similar to one another in shape and size. This two-screen system allows for tight control over the size of the beads, thus allowing the user to modulate the size as needed. Such non-spherical microbeads are disclosed in US 2020/0069846.

### Active Agents

Any of the herein-described gel/hydrogel compositions may be utilized so as to contain an active agent and thereby act as an active agent delivery system when applied to a body surface (e.g., a site of administration) in active agent-transmitting relation thereto. The release of active agents “loaded” into the hydrogel or composite typically involves both absorption of water and desorption of the agent via a swelling-controlled diffusion mechanism. For example, active agent-containing hydrogel compositions may be employed, by way of example, in transdermal drug delivery systems, in topical pharmaceutical formulations, in implanted drug delivery systems, in oral dosage forms, and the like.

Suitable active agents that may be incorporated into the present hydrogel compositions and delivered systemically (e.g., with a transdermal, oral, or other dosage form suitable for systemic administration of a drug) include, but are not limited to: analeptic agents; analgesic agents: anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents: antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine: nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; svmpathomimetics; tranquilizers; and vasodilators including general coronary, peripheral and cerebral. Specific active agents with which the present adhesive compositions are useful include, without limitation, anabasine, capsaicin, isosorbide dinitrate, aminostigmine, nitroglycerine, verapamil, propranolol, silabolin, foridone, clonidine, cytisine, phenazepam, nifedipine, fluacizin, and salbutamol.

For topical drug administration and/or medicated cushions (e.g., medicated footpads), suitable active agents include, by way of example, the following:

Bacteriostatic and bactericidal agents: Suitable bacteriostatic and bactericidal agents include, by way of example: halogen compounds such as iodine, iodopovidone complexes (i.e., complexes of PVP and iodine, also referred to as “povidine” and available under the tradename Betadine from Purdue Frederick), iodide salts, chloramine, chlorohexidine, and sodium hypochlorite; silver and silver-containing compounds such as sulfadiazine, silver protein acetyltannate, silver nitrate, silver acetate, silver lactate, silver sulfate and silver chloride; organotin compounds such as tri-n-butyltin benzoate; zinc and zinc salts; oxidants, such as hydrogen peroxide and potassium permanganate; aryl mercury compounds, such as phenylmercury borate or merbromin; alkyl mercury compounds, such as thiomersal; phenols, such as thymol, o-phenyl phenol, 2-benzyl-4-chlorophenol, hexachlorophen and hexylresorcinol; and organic nitrogen compounds such as 8-hydroxyquinoline, chlorquinaldol, clioquinol, ethacridine, hexetidine, chlorhexedine, and ambazone.

Antibiotic agents: Suitable antibiotic agents include, but are not limited to, antibiotics of the lincomycin family (referring to a class of antibiotic agents originally recovered from Streptomyces lincolnensis), antibiotics of the tetracycline family (referring to a class of antibiotic agents originally recovered from Streptomyces aureofaciens), and sulfur-based antibiotics, i.e., sulfonamides. Exemplary antibiotics of the lincomycin family include lincomycin, clindamycin, related compounds, and pharmacologically acceptable salts and esters thereof. Exemplary antibiotics of the tetracycline family include tetracycline itself, chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Pain relieving agents: Suitable pain relieving agents are local anesthetics, including, but not limited to, acetamidoeugenol, alfadolone acetate, alfaxalone, amucaine, amolanone, amylocaine, benoxinate, betoxycaine, biphenamine, bupivacaine, burethamine, butacaine, butaben, butanilicaine, buthalital, butoxycaine, carticaine. 2-chloroprocaine, cinchocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, .beta.-eucaine, euprocin, fenalcomine, fomocaine, hexobarbital, hexylcaine, hydroxydione, hydroxvprocaine, hydroxytetracaine, isobutyl p-aminobenzoate, kentamine, leucinocaine mesylate, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, and combinations thereof. Tetracaine, lidocaine and prilocaine are referred pain relieving agents herein.

Other topical agents that may be delivered using the present hydrogel compositions as drug delivery systems include the following: antifungal agents such as undecylenic acid, tolnaftate, miconazole, griseofulvine, ketoconazole, ciclopirox, clotrimazole and chloroxylenol; keratolvtic agents, such as salicylic acid, lactic acid and urea; vessicants such as cantharidin; anti-acne agents such as organic peroxides (e.g., benzoyl peroxide), retinoids (e.g., retinoic acid, adapalene, and tazarotene), sulfonamides (e.g., sodium sulfacetamide), resorcinol, corticosteroids (e.g., triamcinolone), alpha-hydroxy acids (e.g., lactic acid and glycolic acid), alpha-keto acids (e.g., glyoxylic acid), and antibacterial agents specifically indicated for the treatment of acne, including azelaic acid, clindamycin, erythromycin, meclocycline, minocycline, nadifloxacin, cephalexin, doxycycline, and ofloxacin; skin-lightening and bleaching agents, such as hydroquinone, kojic acid, glycolic acid and other alpha-hydroxy acids, artocarpin, and certain organic peroxides; agents for treating warts, including salicylic acid, imiquimod, dinitrochlorobenzene, dibutyl squaric acid, podophyllin, podophyllotoxin, cantharidin, trichloroacetic acid, bleomycin, cidofovir, adefovir, and analogs thereof; and anti-inflammatory agents such as corticosteroids and nonsteroidal anti-inflammatory drugs (NSAIDs), where the NSAIDS include ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, and tiaprofenic acid.

For wound dressings, suitable active agents are those useful for the treatment of wounds, and include, but are not limited to bacteriostatic and bactericidal compounds, antibiotic agents, pain relieving agents, vasodilators, tissue-healing enhancing agents, amino acids, proteins, proteolytic enzymes, cytokines, and polypeptide growth factors.

As discussed, in particular embodiments, the hydrogel composition includes a Botulinum neurotoxin (BoNT) particularly a Botulinum toxin type A (BoNTA) material (e.g. Botox or Dysport).

For topical and transdermal administration of some active agents, and in wound dressings, it may be necessary or desirable to incorporate a permeation enhancer into the hydrogel composition in order to enhance the rate of penetration of the agent into or through the skin. Suitable enhancers include, for example, the following: sulfoxides such as dimethylsulfoxide (DMSO) and decylmethylsulfoxide; ethers such as diethylene glycol monoethyl ether (available commercially as Transcutol) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclaza-cycloheptan-2-one (available under the trademark Azone from Nelson Research & Development Co., Irvine, Calif.: see U.S. Pat. Nos. 3,989,816, 4,316,893, 4,405,616 and 4,557,934); alcohols such as ethanol, propanol, octanol, decanol, benzyl alcohol, and the like: fatty acids such as lauric acid, oleic acid and valeric acid: fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Mixtures of two or more enhancers may also be used.

In certain other embodiments, the composite compositions including hydrogel component and nanofibers may also comprise additional optional additive components. Such components are known in the art and can include, for example, fillers, preservatives, pH regulators, softeners, thickeners, pigments, dyes, refractive particles, stabilizers, toughening agents, detackifiers, pharmaceutical agents (e.g., antibiotics, angiogenesis promoters, antifungal agents, immunosuppressing agents, antibodies, and the like), and permeation enhancers. These additives, and amounts thereof, are selected in such a way that they do not significantly interfere with the desired chemical and physical properties of the hydrogel composition.

Absorbent fillers may be advantageously incorporated to control the degree of hydration when the adhesive is on the skin or other body surface. Such fillers can include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, woven and non-woven paper and cotton materials. Other suitable fillers are inert, i.e., substantially non-adsorbent, and include, for example, polyethylenes, polypropylenes, polyurethane polyether amide copolymers, polyesters and polyester copolymers, nylon and rayon.

The compositions can also include one or more preservatives. Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

The compositions may also include pH regulating compounds. Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface.

The compositions may also include suitable softening agents. Suitable softeners include citric acid esters, such as triethylcitrate or acetyl triethylcitrate, tartaric acid esters such as dibutyltartrate, glycerol esters such as glycerol diacetate and glycerol triacetate; phthalic acid esters, such as dibutyl phthalate and diethyl phthalate; and/or hydrophilic surfactants, preferably hydrophilic non-ionic surfactants, such as, for example, partial fatty acid esters of sugars, polyethylene glycol fatty acid esters, polyethylene glycol fatty alcohol ethers, and polyethylene glycol sorbitan-fatty acid esters.

The compositions may also include thickening agents. Preferred thickeners herein are naturally occurring compounds or derivatives thereof, and include, by way of example: collagen; galactomannans; starches; starch derivatives and hydrolysates; cellulose derivatives such as methyl cellulose, hydroxypropylcellulose, hydroxyethyl cellulose, and hydroxypropyl methyl cellulose; colloidal silicic acids; and sugars such as lactose, saccharose, fructose and glucose. Synthetic thickeners such as polyvinyl alcohol, vinylpyrrolidone-vinylacetate-copolymers, polyethylene glycols, and polypropylene glycols may also be used.

In certain embodiments, the hydrogel composite of the invention comprising a hydrogel and nanofibers further comprises a component that promotes angiogenesis. A challenge to achieving clinically relevant soft tissue regeneration prior to the present invention is that the regenerated tissue preferably should be re-vascularized. Therefore, any material that promotes soft tissue regeneration preferably should also encourage angiogenesis. One way to achieve this is through the use of heparin-containing hydrogel components, which can serve as growth factor binding sites to enrich and retain growth factors promoting angiogenesis and tissue formation.

In some embodiments, compositions provided herein further comprise small molecules for delivery, wherein the small molecule is a biologically active material. In some embodiments, the small molecule can cause pharmacological activity or anther direct effect in the diagnosis, cure, mitigation, treatment or prevention of disease or can affect the structure or function of the body.

The gel/hydrogel/nanofiber composites of the invention can also include tissue-repairing agents, such as, a number of growth factors, including epidermal growth factor (EDF), PDGF, and nerve growth factors (NGF's). For example, the compositions may include EGF. Epidermal Growth Factor (EGF) was discovered after the observation that cutaneous wounds in laboratory mice seemed to heal more rapidly when the mice were allowed to lick them. This was not simply due to some antiseptic agent in saliva (such as lysozyme). A specific growth factor, now known as EGF, was shown to be responsible. EGF is identical to urogastrone and has angiogenic properties. Transforming growth factor-alpha (TGFα) is very similar, binding to the same receptor and is even more effective in stimulating epithelial cell regeneration (epithelisation).

Thus, hydrogels including EGF/TGF may advantageously be used in the acceleration of wound healing and burns, reduction in keloid scar formation (especially for burns), skin engraftment dressings, and the treatment of chronic leg ulcers.

Tissue-repairing agents useful in the present invention include a number of growth factors, including epidermal growth factor (EDF), PDGF, and nerve growth factors (NGF's). Generally, growth-promoting hormones will affect between one and four tissues. Many of the products developed from such proteins are targeted towards wound repairs of one kind or another, although there are other indications. Some of the most important tissue growth factors are described further below.

The gel/nanofibers compositions of the invention may also include one or more growth factors that may be useful in the tissue repair methods and other applications of the invention.

The hydrogel/nanofibers compositions of the invention may also include VEGF to promote angiogenesis. Vascular Endothelial Growth Factor (VEGF—also known as vascular permeability factor) is another vascular growth factor that is a multifunctional angiogenic cytokine. It contributes to angiogenesis (blood vessel growth) both indirectly and directly by stimulating proliferation of endothelial cells at the microvessel level, causing them to migrate and to alter their generic expression. VEGF also makes theses endothelial cells hyperpermeable, causing them to release plasma proteins outside the vascular space, which causes changes in the area, contributing to angiogenesis.

The compositions of the invention may also include FGF. Fibroblast Growth Factor (FGF) is actually a family of at least 19 14 18 kD peptides belonging to the heparin-binding growth factors family and are mitogenic for cultured fibroblasts and vascular endothelial cells. They are also angiogenic in vivo and this angiogenicity is enhanced by TNF. FGF's may be used in a similar manner to EGF, bFGF, also known as FGF-2, is involved in controlling human megakaryocytopoiesis and FGFs have been shown to be effective in stimulating endothelial cell formation, and in assisting in connective tissue repair.

Hydrogel/nanofibers compositions may also comprise Keratinocyte Growth Factor (KGF), also known as FGF-7, for use in wound healing and other disorders involving epithelial cell destruction.

Transforming Growth Factors (TGF's) have the ability to transform various cell lines, and can confer, for example, the ability to grow in culture for more than a limited number of generations, growth in multiple layers rather than monolayers, and the acquisition of an abnormal karyotype. There are at least five members of the TGF family, the two most widely studied being TGF-alpha and TGF-beta. The former is mitogenic for fibroblasts and endothelial cells, angiogenic, and promotes bone resorption. Compositions also may include TGF. TGF-beta is a general mediator of cell regulation, a powerful inhibitor of cell growth, and inhibits the proliferation of many cell types. TGF-beta can antagonize the mitogenic effects of other peptide growth factors and can also inhibit the growth of many tumour cell lines. TGF-beta also has angiogenic effects and promotes collagen formation in fibroblasts.

Hydrogel/nanofiber compositions of the present invention may usefully comprise collagen, for example. Although collagen, in this form, is unlikely to serve a useful structural function, it primarily serves as a sacrificial protein where proteolytic activity is undesirably high, thereby helping to prevent maceration of healthy tissue, for example.

Hydrogel/nanofiber compositions can also include certain enzymes. Enzymes are used in the debridement of both acute and chronic wounds. Debridement is the removal of nonviable tissue and foreign matter from a wound and is a naturally occurring event in the wound-repair process. During the inflammatory phase, neutrophils and macrophages digest and remove “used” platelets, cellular debris, and avascular injured tissue from the wound area. However, with the accumulation of significant amounts of damaged tissue, this natural process becomes overwhelmed and insufficient. Build-up of necrotic tissue then places considerable phagocytic demand on the wound and retards wound healing. Consequently, debridement of necrotic tissue is a particular objective of topical therapy and an important component of optimal wound management.

Enzymes, for example, may be incorporated into hydrogels of the present invention for topical application to provide a selective method of debridement. Suitable enzymes may be derived from various sources, such as krill, crab, papaya, bovine extract, and bacteria Commercially available, suitable enzymes include collagenase, papain/urea, and a fibrinolysin and deoxyribonuclease combination.

Enzymes for use in the present invention generally work in one of two ways: by directly digesting the components of slough (e.g., fibrin, bacteria, leukocytes, cell debris, serous exudate, DNA); or, by dissolving the collagen “anchors” that secure the avascular tissue to the underlying wound bed.

Hydrogels of the present invention may comprise Dakin's solution, if desired, generally to exert antimicrobial effects and odor control. As a debridement agent, Dakin's solution is non-selective because of its cytotoxic properties. Dakin's solution denatures protein, rendering it more easily removed from the wound. Loosening of the slough also facilitates debridement by other methods. Hydrogels comprising Dakin's solution may be changed twice daily if the goal is debridement. Periwound skin protection should generally be provided with ointments, liquid skin barrier film dressings, or solid skin barrier wafers, for example.

The gel of the present invention may be delivered by any suitable method, such as via a syringe or bellows pack (single dose delivery systems) or a multidose system, such as a pressurized delivery system or delivery via a ‘bag in the can’ type system (such as that published in WO98/32675). An example of a bellows pack is shown in published UK design number 2082665.

As such, the present invention also extends to a single dose delivery system comprising a gel according to the present invention, for the treatment of wounds. The invention also extends to a pressurized delivery system comprising a gel according to the present invention, and a pressurized hydrogel according to the present invention in an aerosol container capable of forming a spray upon release of pressure therefrom. Use of such delivery means allows the gel to be delivered to areas on a patient which are otherwise difficult to reach by direct application.

In certain embodiment, it may be advantageous to render the hydrogel compositions of the invention electrically conductive for use in biomedical electrodes and other electrotherapy contexts, i.e., to attach an electrode or other electrically conductive member to the body surface. For example, the hydrogel composition may be used to attach a transcutaneous nerve stimulation electrode, an electrosurgical return electrode, or an EKG electrode to a patient's skin or mucosal tissue. These applications involve modification of the hydrogel composition so as to contain a conductive species. Suitable conductive species are ionically conductive electrolytes, particularly those that are normally used in the manufacture of conductive adhesives used for application to the skin or other body surface, and include ionizable inorganic salts, organic compounds, or combinations of both. Examples of ionically conductive electrolytes include, but are not limited to, ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, and redox couples such as a mixture of ferric and ferrous salts such as sulfates and gluconates. Preferred salts are potassium chloride, sodium chloride, magnesium sulfate, and magnesium acetate, and potassium chloride is most preferred for EKG applications. Although virtually any amount of electrolyte may be present in the adhesive compositions of the invention, it is preferable that any electrolyte present be at a concentration in the range of about 0.1 to about 15 wt. % of the hydrogel composition. The procedure described in U.S. Pat. No. 5,846,558 to Nielsen et al. for fabricating biomedical electrodes may be adapted for use with the hydrogel compositions of the invention, and the disclosure of that patent is incorporated by reference with respect to manufacturing details. Other suitable fabrication procedures may be used as well, as will be appreciated by those skilled in the art.

### Nanofibers

The nanofibers may include, but not limited to, nanofibers, nanotubes, nanofilaments, mesh sections, branched filaments or networks. The nanofibers may also comprise any suitable chemical functional groups to facilitate the covalent or noncovalent crosslinking between the nanofibers and the polymers of the hydrogels of the invention. Method, techniques, and materials are well known in the art for making and functionalizing nanofibers.

In certain embodiments, microfabrication methods are used to make the nanofibers. In various embodiments, the disclosed devices can be assembled and/or manufactured using any suitable microfabrication technique. Such methods and techniques are widely known in the art.

The nanofibers may also be fabricated by electrostatic spinning (also referred to as electrospinning). The technique of electrospinning of liquids and/or solutions capable of forming fibers, is well known and has been described in a number of patents, such as, for example, U.S. Pat. Nos. 4,043,331 and 5,522,879. The process of electrospinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening).

The process of electrostatic spinning has typically been directed toward the use of the fibers to create a mat or other non-woven material, as disclosed, for example, in U.S. Pat. No. 4,043,331. Nanofibers ranging from 50 nm to 5 micrometers in diameter can be electrospun into a nonwoven or an aligned nanofiber mesh. Due to the small fiber diameters, electrospun textiles inherently possess a very high surface area and a small pore size. These properties make electrospun fabrics potential candidates for a number of applications including: membranes, tissue scaffolding, and other biomedical applications.

Electrostatically spun fibers can be produced having very thin diameters. Parameters that influence the diameter, consistency, and uniformity of the electrospun fibers include the polymeric material and cross-linker concentration (loading) in the fiber-forming combination, the applied voltage, and needle collector distance. According to one embodiment of the present invention, a nanofiber has a diameter ranging from about 1 nm to about 100 mm. In other embodiments, the nanofiber has a diameter in a range of about 1 nm to about 1000 nm. Further, the nanofiber may have an aspect ratio in a range of at least about 10 to about at least 100. It will be appreciated that, because of the very small diameter of the fibers, the fibers have a high surface area per unit of mass. This high surface area to mass ratio permits fiber-forming solutions or liquids to be transformed from liquid or solvated fiber-forming materials to solid nanofibers in fractions of a second.

The polymeric material used to form the nanofibers/nanostructures of the invention may be selected from any fiber forming material which is compatible with the cross-linking agents. Depending upon the intended application, the fiber-forming polymeric material may be hydrophilic, hydrophobic or amphiphilic. Additionally, the fiber-forming polymeric material may be a thermally responsive polymeric material.

Synthetic or natural, biodegradable or non-biodegradable polymers may form the nanofibers/nanostructures of the invention. A “synthetic polymer” refers to a polymer that is synthetically prepared and that includes non-naturally occurring monomeric units. For example, a synthetic polymer can include non-natural monomeric units such as acrylate or acrylamide units. Synthetic polymers are typically formed by traditional polymerization reactions, such as addition, condensation, or free-radical polymerizations. Synthetic polymers can also include those having natural monomeric units, such as naturally-occurring peptide, nucleotide, and saccharide monomeric units in combination with non-natural monomeric units (for example synthetic peptide, nucleotide, and saccharide derivatives). These types of synthetic polymers can be produced by standard synthetic techniques, such as by solid phase synthesis, or recombinantly, when allowed.

A “natural polymer” refers to a polymer that is either naturally, recombinantly, or synthetically prepared and that consists of naturally occurring monomeric units in the polymeric backbone. In some cases, the natural polymer may be modified, processed, derivatized, or otherwise treated to change the chemical and/or physical properties of the natural polymer. In these instances, the term “natural polymer” will be modified to reflect the change to the natural polymer (for example, a “derivatized natural polymer”, or a “deglycosylated natural polymer”).

Nanofiber materials, for example, may include both addition polymer and condensation polymer materials such as polyolefin, polyacetal, polyamide, polyester, cellulose ether and ester, polyalkylene sulfide, polyarylene oxide, polysulfone, modified polysulfone polymers and mixtures thereof. Exemplary materials within these generic classes include polyethylene, poly(ε-caprolactone), poly(lactate), poly(glycolate), polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms. Exemplary addition polymers tend to be glassy (a Tg greater than room temperature). This is the case for polyvinylchloride and polymethylmethacrylate, polystyrene polymer compositions, or alloys or low in crystallinity for polyvinylidene fluoride and polyvinyl alcohol materials.

Nanofibers can also be formed from polymeric compositions comprising two or more polymeric materials in polymer admixture, alloy format, or in a crosslinked chemically bonded structure. Two related polymer materials can be blended to provide the nanofiber with beneficial properties.

Biodegradable polymers can also be used in the preparation of the nanofibers of the invention. Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with, for example, implantable medical devices include polylactide, polyglycolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used.

The inclusion of cross-linking agents within the composition forming the nanofiber, allows the nanofiber to be compatible with a wide range of support surfaces. The cross-linking agents can be used alone or in combination with other materials to provide a desired surface characteristic.

Suitable cross-linking agents include either monomeric (small molecule materials) or polymeric materials having at least two latent reactive activatable groups that are capable of forming covalent bonds with other materials when subjected to a source of energy such as radiation, electrical or thermal energy. In general, latent reactive activatable groups are chemical entities that respond to specific applied external energy or stimuli to generate active species with resultant covalent bonding to an adjacent chemical structure. Latent reactive groups are those groups that retain their covalent bonds under storage conditions but that form covalent bonds with other molecules upon activation by an external energy source. In some embodiments, latent reactive groups form active species such as free radicals. These free radicals may include nitrenes, carbine or excited states of ketones upon absorption of externally applied electric, electrochemical or thermal energy. Various examples of known or commercially available latent reactive groups are reported in U.S. Pat. Nos. 4,973,493; 5,258,041; 5,563,056; 5,637,460; or 6,278,018.

For example, the commercially available multifunctional photocrosslinkers based on trichloromethyl triazine available either from Aldrich Chemicals, Produits Chimiques Auxiliaires et de Syntheses, (Longjumeau, France), Shin-Nakamara Chemical, Midori Chemicals Co., Ltd. or Panchim S. A. (France) can be used. The eight compounds include 2,4,6-tris(trichloromethyl)-1,3,5 triazine, 2-(methyl)-4,6-bis(trichloromethyl)-1,3,5-triazine, 2-(4-methoxynaphthyl)-4,6-bis(trichloromethyl)-1,3,5-triazine, 2-(4-ethoxynaphthyl)-4,6-bis(trichloromethyl)-1,3,5-triazine, 4-(4-carboxylphenyl)-2,6-bis(trichloromethyl)-1,3,5-triazine, 2-(4-methoxyphenyl)-4,6-bis(trichloromethyl)-1,3,5-triazine, 2-(1-ethen-2-2′-furyl)-4,6-bis(trichloromethyl)-1,3,5-triazine and 2-(4-methoxystyryl)-4,6-bis(trichloromethyl)-1,3,5-triazine.

### Methods of Use

The nanofibers-hydrogel composite disclosed herein can be used advantageously in treating neural tissues, for example, as being applied on the neurosurgical site or wound as an implant or device. The nanofibers-hydrogel composite may also be used to deliver additional active agents described herein, such as antibiotics, growth factors, and immunosuppressive agents.

In an aspect, the composite is for use in treating neural tissues in a subject. The method includes applying the hydrogel composite as described herein to or around the neural tissues.

In certain embodiments, the neural tissues includes peripheral nerves (e.g., limb or muscle nerves).

In some embodiments, the subject is treated with neurosurgery. For example, the subject may be a patient who had neurosurgical treatment and was further treated with nerve decompression and/or neurolysis.

The hydrogel composite as used in the treatment is capable of at least one of i) suppressing scarring, ii) suppressing adhesion of the neural tissues, and/or iii) promoting regeneration of the neural tissue in the subject.

In some embodiments, the hydrogel composite is injected or implanted on or around the neural tissues, or other targeted treatment site. Alternatively, the hydrogel composite is applied for dermal or subdermal administration into the neural tissues or other targeted treatment site of the subject.

Because the hydrogel composite also suitably may include other components (e.g., growth factors, compounds stimulating angiogenesis, immunomodulators, inhibitors of inflammation, and combinations thereof) as described herein, the treatment may be effective in inhibition of the growth of tumour cells, stimulation of angiogenesis, modulating immune responses, inhibiting inflammation, and the like. Further, the hydrogel composite also the hydrogel composite may include one or more compounds that have therapeutic effects, vascularization effects, anti-vascularization effects, anti-inflammatory effects, anti-bacterial effects, antihistamine effects, and combinations thereof.

In an aspect, provided herein is an implant or a device including the implant for treating a subject after neurosurgery. The implant includes the hydrogel composite as described herein. The implant may be particularly used for treating neural tissues (e.g., peripheral nerves) when the subject had neurosurgical procedure, nerve decompression and/or neurolysis.

Also provided is a kit including the implant as described herein and an applicator, for example, the applicator may be an injection syringe.

In some embodiments, the implant may be dehydrated or dried, e.g., for storage purpose. In certain embodiments, the kit may include a vial including water, saline solution or suitable fluid for reconstitution of the dehydrated implant.

Specifically preferred hydrogel compostions for administration in accordance with the present methods and kits are disclosed in the Examples which follow, including Examples 1, 4 and 5. Specifically preferred hydrogel compostions for administration in accordance with the present methods and kits are also disclosed in US 2020/0069846 (including Example 12 of US 2020/0069846). Specifically preferred hydrogel compostions for administration in accordance with the present methods and kits are also disclosed in U.S. Pat. Nos. 10,471,181 and 11,684,700.

Preferred dosage amounts are exemplified in the examples which follow, as well as the dosages set forth in in US 2020/0069846 including the examples thereof. Preferred dosage amounts are also disclosed in U.S. Pat. Nos. 10,471,181 and 11,684,700. Optimal dosages also can be readily determined empirically, including through in vivo models or with specific patient evaluations.

Although particular examples and uses for the hydrogel/nanostructure composites of the invention have been described herein, such specific uses are not meant to be limiting. The hydrogel/nanostructure composites of the invention can be used for any application generally used for known hydrogels, and in particular, are useful for the repair and/or regeneration of soft tissue anywhere in the body.

### EXAMPLES

### Example 1: Preparation of Hydrogel Composite

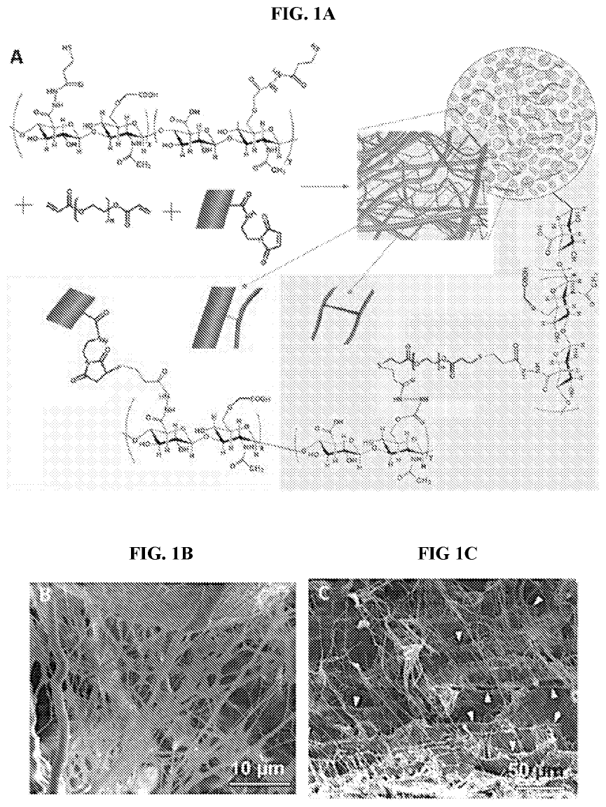

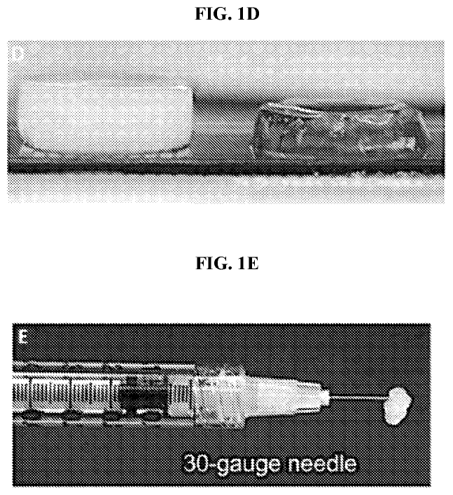

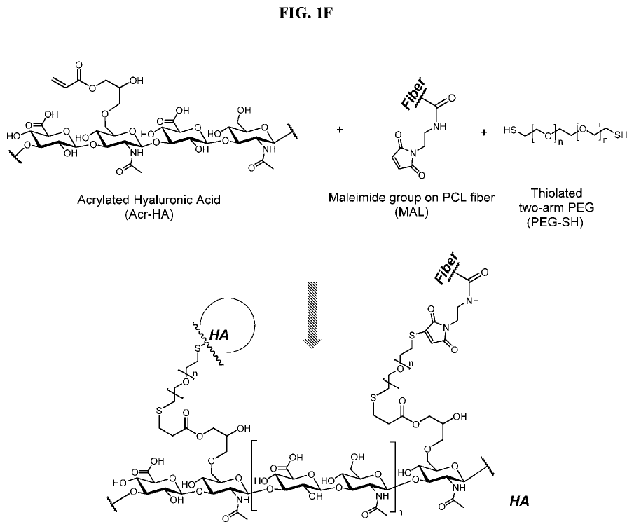

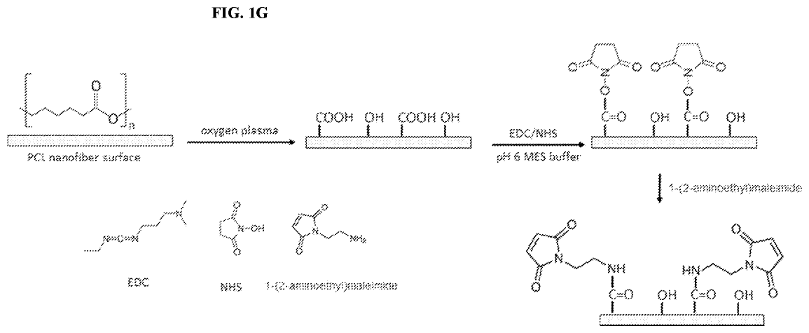

Firstly, carboxyl groups were introduced to polycaprolactone (PCL) fiber surface through plasma activation. Then, a fraction (e.g., 1 to 3%) of the carboxyl group was converted to the thiol-reactive maleimide (MAL) group to generate MAL-functionalized PCL (MAL-PCL) fibers (FIG. 1G). MAL-PCL fibers were then fragmented in a cryogenic milling chamber filled with liquid nitrogen. The average length of the fibers was controlled within the range of 20 to 100 m by adjusting the duration of the cooling and milling cycles. The MAL-PCL fiber fragments were then mixed with the HA hydrogel precursors (thiolated HA) at predetermined ratios to form composite (FIG. 1F). During the gelation process, MAL-PCL fiber fragments were conjugated to HA-SH cross-linked network, forming interfacial covalent bonds and then generating an integrated composite structure (FIG. TA). Scanning electron microscopy images revealed that these fibers were connected to the dried HA hydrogel network, exhibiting a fibrillar microarchitecture similar to what was observed in native adipose tissue ECM (FIGS. 1, B and C). A marked reinforcement effect was observed when these fibers were bonded in the HA hydrogel matrix (FIG. 1D).

Furthermore, the composite component premix could readily pass through a 30-gauge needle within 30 min after mixing (FIG. 1E). The passage through the needle did not induce separation of the fibers from the hydrogel phase nor did it affect gelation kinetics. After complete gelation, PCL fiber fragments were distributed relatively evenly throughout the hydrogel.

### Example 2: Use of a Nanofiber Hydrogel Composite for Perineural Adhesion Prevention in a Rodent Model

Perineural adhesions can form after any surgical intervention involving peripheral nerves. Adhesion formation may then lead to nerve entrapment and compressive neuropathy, which can result in a wide variety of symptoms ranging from sensory deficits to motor weakness. The poly(s-caprolactone) (PCL) nanofiber/hyaluronic acid hydrogel composite prepared in Example 1 was used to evaluate its effect of reducing perineural adhesion formation in a rodent hindlimb model.

Methods: This study was performed with Institutional Animal Care and Use Committee approval. Male Lewis rats underwent bilateral circumferential mechanical irritation of the sciatic nerve to induce adhesion formation with subsequent primary closure. Animals then underwent a secondary neurolysis 8 weeks post-operatively. At the time of neurolysis, the experimental group (n=6) were treated with circumferentially application of NHC around the sciatic nerve before closure and the control group were closed without treatment (n=6). Both groups were sacrificed 8 weeks after their secondary surgery. At the time of euthanasia, all rodents underwent unilateral biomechanical force testing to assess the breaking point of the perineural adhesions surrounding the sciatic nerve (measured in Newtons). In the contralateral limb, the sciatic nerve, surrounding muscle, and NHC in experimental animals was harvested to assess perineural collagen deposition using hematoxylin and eosin (H&E) and Masson's Trichrome staining.

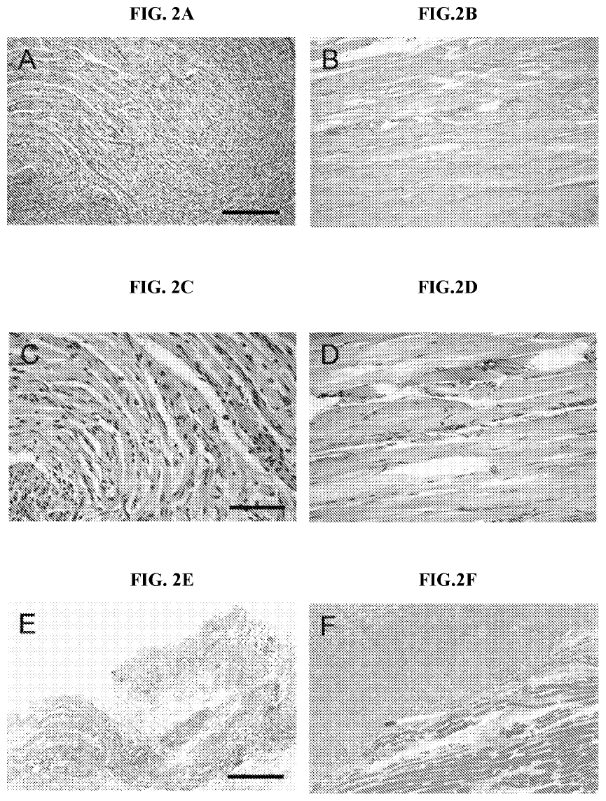

Results: Significant perineural adhesions were visually apparent after sciatic nerve irritation in the control group. In the experimental group, the sciatic nerve was grossly encapsulated by the NHC which closely resembled subcutaneous fat with visible neovascularization. Biomechanical testing demonstrated the average force required to remove the nerve from the wound bed in the experimental group was 2.02±0.43 N. In the control group, the sciatic nerve could not be removed from the wound bed and the average force prior to failure was 2.77±0.18 N. Collagen deposition, a measure of scar formation around the sciatic nerve, was assessed via H&E and MT staining (FIG. 2). Minimal collagen deposition (stained blue) was seen in the experimental group compared to control, indicating a decrease in scar formation in the animals treated with perineural application of the NHC.

### Example 3: Preparation and Analysis of Additional Hydrogel Composite

### Nanofiber Hydrogel Composite

A material of Example 1 was prepared and analyzed.

Sodium hyaluronate with molecular weight of 1.5 MDa (HA, research grade) was purchased from LifeCore Biomedical Inc. (Chaska, MN, USA). Glycidyl acrylate (GA) was obtained from TCI America Inc. (Portland, OR, USA). The poly (ethylene glycol) dithiol (HS-PEG-SH) with an average MW of 5 kDa (PEG-SH, MW 5 kDa) was from JenKem Technology (Plano, TX, USA). All other chemical reagents were purchased from Sigma-Aldrich (St. Louis. MO, USA) unless otherwise noted.

### NHC Microgel Histology and Imaging

HA microgels were firstly activated by 50 mM EDC-HCl (ProteoChem) and 20 mM NHS (Sigma Aldrich) in 0.1M MES buffer (Sigma Aldrich) for 30 minutes. Then, after washing the microgels with PBS for 3 times, microgels were added to 0.1 mg/mL FITC-labeled bovine albumin (Thermo Fisher) for 2 hours for conjugation. After the conjugation, HA microgels were washed with PBS for another 3 times to remove the excessive bovine albumin. 100 μL solution containing microgels then was added to glass slide and covered by cover slip and dried at 4° C. for 2 hours. Microgel slides were then imaged by ZEISS Apotome 3 Microscope to determine the shape and morphology of HA microgels.

### Mechanical Properties of NHC Microgels

Rheological properties of microgels were determined using a parallel plate rheometer (AR2, TA Instruments) at 25° C. Samples were subjected to strain sweeps at a frequency of 1 Hz to determine the linear viscoelastic region. Storage modulus (G′) and loss modulus (G″) were calculated from the resulting data.

### Imaging and Characterization of the Micro-Structures of Fibers and NHCs

Scanning Electron Microscopy (SEM) was performed using Thermo Scientific Helios G4 UC FIBSEM. Briefly, for the fiber SEM imaging, the electrospun fiber mat was sliced gently using razor blade, and stuck to the SEM stubs, the samples then were imaged under the In-Beam SEM mode for high resolution imaging. Similarly, NHC samples were firstly lyophilized and sliced in a similar manner to keep the sample intact, and then imaging was performed. Based on 5 randomly selected SEM images, the fiber diameters and pore sizes were analyzed by an image analysis model (ImageJ. NIH). Quantitative analysis was then conducted.

### In Vitro Degradation Test of NHCs

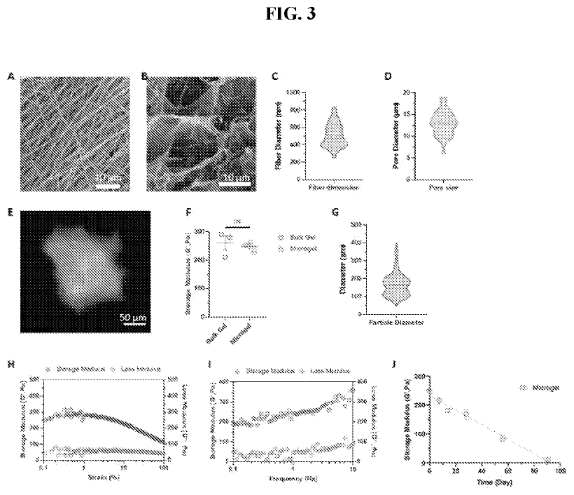

In vitro degradation test was performed in an 37° C. incubator (Thermo Fisher) to mimic the in vivo conditions. Briefly, after fabricating 3 NHC samples, they were stored in a sterilized syringe, and maintained in a 37° C. incubator. At day 0, 7, 14, 28 and 56, 3 independent samples were taken out sterilely from the syringe to perform the rheological test (AR2, TA Instruments). The storage moduli (G′) of those samples were measured to represent the degradation or the stability of the NHCs (FIG. 3).

### Example 4: In Vivo Functional Testing

Adult, male Lewis rats, aged 14 to 16 weeks, (Charles River Laboratories, Wilmington, MA) were used in this study. All experimental surgeries, postoperative care, and behavioral testing were approved by the Johns Hopkins University Animal Care and Use Committee (ACUC Protocol Number: RA21M309). Animals were given one week to acclimate to the facilities prior to undergoing surgical procedures. Rats were randomized into primary and secondary nerve adhesion cohorts as outlined below.

### General Preoperative and Postoperative Care

General anesthesia was induced using 2.5% isoflurane in 100% oxygen, then anesthesia was maintained with 1.5-2.0% isoflurane in oxygen using a rodent nose cone. The surgical site was clipped and prepared using standard aseptic technique. Rats were placed in lateral decubitus, with the lateral aspect of the operative hindlimb shaved of all fur. The base of the tail was taped to the surgery table to eliminate movement from manipulation during surgery. All nerve transection surgeries were carried out using a stereo microscope adapted for surgical use (Leica Microsystems, Buffalo Grove, IL). All animals received subcutaneous buprenorphine (0.1 mg/kg) analgesia and enrofloxacin (1 mg/kg) prophylaxis postoperatively.

### Sciatic Nerve Exposure, Neurolysis and Mechanical Irritation of Surrounding Wound Bed

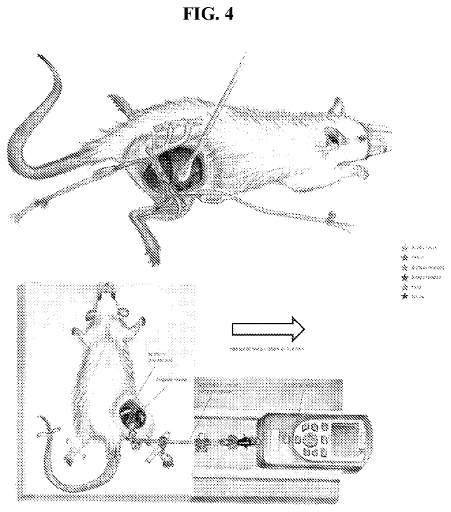

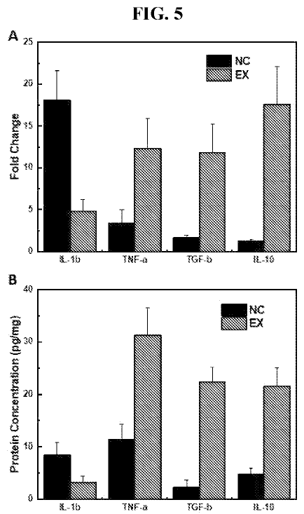

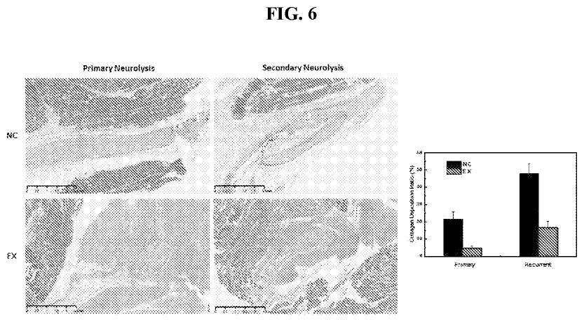

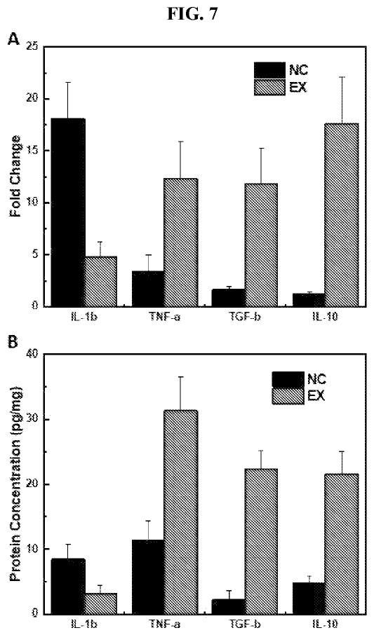

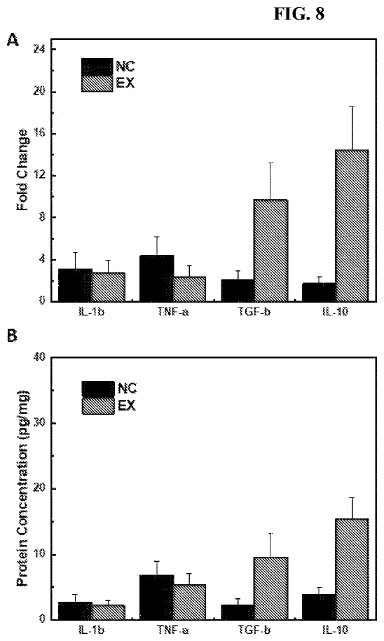

A skin incision was made parallel and just inferior to the femur and spanning the length of the femur. The fascia lata was incised and the biceps femoris muscle was elevated away from the femur and retracted posteriorly with Lonestar retractors (eSutures, Mokena. IL). Blunt dissection was then used to approach the sciatic nerve deep and posterior to the biceps femoris. The sciatic nerve and its terminal branches were circumferentially neurolysed (FIG. 4).

### Primary Neurolysis Cohort

The primary neurolysis cohorts underwent bilateral circumferential mechanical irritation of the sciatic nerve with a sterile cotton swab to induce adhesion formation. The experimental group (n=6) was treated with circumferential application of NHC (500 microL) around the sciatic nerve before closure; the control group (n=6) was closed without NHC application. After eight weeks animals underwent endpoint analysis using; 1) biomechanical testing, afterwards the nerve and surrounding wound bed were harvested for 2) histological assessment and 3) inflammatory cytokine gene expression, then animals were sacrificed.

### Secondary Neurolysis Cohort

The secondary neurolysis cohorts underwent the same primary surgery as described above, though no animals received treatment during primary sciatic nerve neurolysis and mechanical irritation. 8 weeks later, animals underwent re-exposure and neurolysis of the sciatic nerve with mechanical irritation. At the time of the secondary procedure, the experimental group (n=6) were treated with circumferentially application of NHC around the sciatic nerve before closure and the control group were closed without treatment (n=6). Both groups underwent the same endpoint analysis 8 weeks after their secondary surgery, and then were sacrificed (FIG. 6).

### Biomechanical Testing

Upon reaching their endpoints and prior to euthanasia, all rodents underwent unilateral biomechanical force testing to assess the breaking point of the perineural adhesions surrounding the sciatic nerve (measured in Newtons) in a procedure adapted from previous, peer-reviewed methodologies (16, 17). The sciatic nerve was identified, and transected distal to the area of perineural scar formation, approximately 1 mm proximal to the trifurcation into the common peroneal, sural, and tibial nerves. An alligator clamp was then attached to this distal end of the sciatic nerve; the other end of the alligator clamp was then connected to a force transducer (FIG. 4). Proximally, the sciatic nerve was identified at the sciatic notch and was then transected proximally. The force transducer was then translocated horizontally at a rate of 3 cm/min until final failure of the perineural adhesions around the sciatic nerve or until nerve rupture and pullout from the alligator clamp.

### Histology

### Hematoxylin & Eosin Staining

To assess the nerve cross-section area, hematoxylin and eosin staining of the target organ was performed. We used Hematoxylin 7211 and Eosin-Y, both from Thermo Scientific™ (Waltham, MA. USA), using standard manufacturer protocol.

### Masson's Trichrome Collagen Staining

To analyze collagen deposition within the NHC. Masson's Trichrome staining was performed on 8 m frozen tissue sections using the Stain Kit from Polysciences, Inc. (Warrington, PA) according to the manufacturer's instructions. Briefly, sections were initially incubated in Bouin's solution (overnight at room temperature). After washing with tap water for 1-2 min, the sections were stained with hematoxylin's working solution for 10 min, then washed with running tap water followed by distilled water. The sections were then incubated with Biebrich scarlet acid fuchsin solution for 5 min, phosphotungstic/phosphomolybdic acid for 10 min, aniline blue for 5 min, acetic acid (1%) for 1 min, and finally mounted on slides; distilled water rinses were performed in between each step. The sections were analyzed. An image analysis system (Image J, NIH) was used to outline the injected material, and to calculate the percentage area of collagen staining.

### Quantitative RT-PCR for Inflammatory Cytokine Gene Expression

To study the dynamics of pro- and anti-inflammatory cytokines in the wound bed, mRNA expression of TNF-α, IL-1β. TGF-β1 and IL-10 was quantified using quantitative RT-PCR (qRT-PCR). Total RNA from snap-frozen tissue and NHC was isolated using Trizol reagent (Life Technologies, Grand Island, NY), and then purified using the RNeasy mini kit (Qiagen, Valencia. CA). The RNA integrity was evaluated with an Agilent 2100 Bioanalyzer (Agilent Technologies. Palo Alto, CA), and concentration was measured using a Nanodrop 8000 spectrophotometer (NanoDrop Products, Wilmington, DE). cDNA was generated using the Superscript III First Strand synthesis system for RT-PCR with Oligo(dT) primers, according to the manufacturer's instructions (Invitrogen, California, USA). qRT-PCR was performed with TaqMan probes and primers using QuantStudio™ 12 K Flex Real-Time PCR System (Life Technologies). Briefly, 1 mg of total RNA from each sample was reverse transcribed in a 20 ml reaction using SuperScript® III First-Strand cDNA synthesis kit (Life Technologies). After optimized dilution of the resulting cDNA, PCR reaction was carried out in 20 mL reaction volumes containing diluted cDNA (20 ng RNA input) and gene-specific probes/primers as per manufacturer's protocol. An average threshold-cycle (Ct) from triplicate assays was used to determine the GAPDH-normalized gene expression.

### ELISA for Inflammatory Cytokine Protein Concentration

A segment of the sciatic nerve was snap-frozen at the time of initial harvest and was eventually placed in round bottom microfuge tubes on ice. 300 μL complete extraction buffer (100 mM Tris, pH 7.4, 150 mM NaCl, 1 mM egtazic acid, 1 mM ethylenediaminetetraacetic acid, 1% Triton X-100, 0.5% sodium deoxycholate, phosphatase inhibitor cocktail (1 mL, 50× stock), protease inhibitor cocktail (with phenyl methyl sulfonyl fluoride, Abcam, 250 μL, 500× stock) was added to each 5-mg piece of tissue before homogenization and shaking at 4° C. for 2 h. The suspensions were combined and centrifuged for 20 min at 13000 rpm at 4° C. and placed on ice. The supernatant was aliquoted to a chilled tube for ELISA (DuoSet) according to the manufacturer's protocols.

### Results

### Biomechanical Testing

Table 1 below shows the results of biomechanical testing on the sciatic nerve-wound bed interface in animals that underwent mechanical irritation without NHC treatment at 0, 2, 4, 6, and 8 weeks post-operatively. Notably, at the 6- and 8-week timepoints, the sciatic nerve could not be removed from the surrounding wound bed due to significant perineural adhesion formation and the average force prior to nerve rupture was 1.67±0.40 and 3.14±0.70, respectively.

TABLE 1
Biomechanical Characterization of Perineural
Scar Formation at Various Timepoints

| Time (weeks) | Average Force (N) |
|---|---|
| 0 | 0.1675 ± 0.1159* |
| 2 | 0.4825 ± 0.1725* |
| 4 | 0.6525 ± 0.2654 |
| 6 | 1.6650 ± 0.4037\hat{ } |
| 8 | 3.1375 ± 0.6998\hat{ } |

*Average force required for removal of nerve from wound bed;
\hat{ }Average force prior to nerve rupture

In the primary neurolysis cohort, perineural adhesions were visually apparent after mechanical irritation of the sciatic nerve in the control group. In the experimental group, the sciatic nerve was encapsulated within the NHC, with some ingrowth of de novo adipose tissue (FIG. 6). Biomechanical testing demonstrated that the average force required to remove the nerve from the wound bed in the experimental group was 0.69±0.11 N. In the control group, the sciatic nerve could not be removed from the wound bed and the average force prior to nerve rupture was 2.98±0.57 N.

In the secondary neurolysis cohort, significant perineural adhesions were present 8 weeks after the initial procedure involving mechanical irritation of the sciatic nerve in both the control and experimental animals. In the control group, adhesions were noted 8 weeks after neurolysis and closure; whereas in the experimental group, the sciatic nerve was grossly encapsulated by the NHC, which closely resembled adipose tissue with visible neovascularization (FIG. 6). Biomechanical testing demonstrated the average force required to remove the nerve from the wound bed in the experimental group was 2.02±0.43 N. In the control group, the sciatic nerve could not be removed from the wound bed and the average maximum force prior to rupture of the nerve was 2.77±0.18 N.

### Histology and Collagen Deposition Quantification

Hematoxylin and eosin and Masson's trichome staining was performed on tissue samples harvested from the animals at the time of euthanasia. Longitudinal cross-sections of the interface between the sciatic nerve and the surrounding muscle were examined to identify the degree of collagen deposition and scar formation (FIG. 6). In the primary neurolysis cohort, the average percentage of collagen deposition surrounding the nerve was 21.4±4.4% and 4.6±1.4% in the control and experimental groups respectively (p<0.0001). In the secondary neurolysis cohort, the average percentage of collagen deposition surrounding the nerve was 47.8±5.8% and 16.7±3.7% in the control and experimental groups respectively (p<0.0001).

### Genomic and Proteomic Analysis of Wound-Healing Environment at Nerve-Muscle Interface

We examined whether the NHC mediated wound healing and the gene expression of pro- and anti-inflammatory markers using a PCR array. Furthermore, we performed an analysis of the protein concentration of these cytokines at the nerve-muscle interface using ELISA.

In the primary neurolysis group, animals treated with the NHC had a substantial up-regulation of anti-inflammatory cytokines, namely TGF-β1 and IL-10 (18). In the experimental animals, there was an average 11.8-fold increase in TGF-β1 and 17.6-fold increase in IL-10 compared to the 1.6-fold and 1.2-fold increase seen in untreated controls, at 8 weeks post-operatively (FIG. 7). Experimental animals also showed an average 12.3-fold increase in TNF-α, a cytokine produced by activated macrophages with both pro-inflammatory and immune-modulatory effects (18). Conversely, control animals had an 18.1-fold increase in expression of IL-1b, a potent pro-inflammatory cytokine associated with host-defense immune responses to infection and injury (19, 20). Animals treated with the NHC showed a significant increase in the protein concentration of TGF-β1 and IL-10 compared to untreated controls (p<0.001); control animals had elevated levels of IL-1b compared to experimental animals (p=0.0008) (FIG. 7).

In the secondary neurolysis group, animals treated with the NHC had an upregulation of TGF-β1 and IL-10 gene expression, with a 9.7-fold and 14.4-fold increase respectively, at 8 weeks after application of the NHC and 16 weeks after initial mechanical irritation of the sciatic nerve (FIG. 8). Gene expression of IL-1b and TNF-α were elevated in the control animals (3.1-fold and 4.4-fold increase). Animals treated with the NHC showed a significant increase in the protein concentration of TGF-β1 and IL-10 compared to untreated controls (p=0.007 and p<0.0001, respectively) (FIG. 8).

### REFERENCES

1. Mazal P R, Millesi H. Neurolysis: is it beneficial or harmful? Acta Neurochir Suppl. 2005:92:3-6.
2. Crosio A, Valdatta L, Cherubino M, Izzo M, Pellegatta I, Pascal D, et al. A simple and reliable method to perform biomechanical evaluation of postoperative nerve adhesions. J Neurosci Methods. 2014; 233:73-7.
3. Thatte M R, Mansukhani K A. Compressive neuropathy in the upper limb. Indian J Plast Surg. 2011; 44(2):283-97.
4. Wilgis E F, Murphy R. The significance of longitudinal excursion in peripheral nerves. Hand Clin. 1986; 2(4):761-6.
5. Millesi H, Rath T, Reihsner R. Zoch G. Microsurgical neurolysis: its anatomical and physiological basis and its classification. Microsurgery. 1993; 14(7):430-9.
6. Tos P, Crosio A, Pugliese P. Adani R. Toia F. Artiaco S. Painful scar neuropathy: principles of diagnosis and treatment. Plastic and Aesthetic Research. 2015; 2:156-64.
7. Isaacs J, McMurtrv J. Different nerve grafting and wrapping options in upper extremity surgery. Current Orthopaedic Practice. 2014; 25(5):456-61.
8. De Smet L. Vandeputte G. Pedicled fat flap coverage of the median nerve after failed carpal tunnel decompression. J Hand Surg Br. 2002; 27(4):350-3.
9. MacKay M A, Fischgrund J S, Herkowitz H N, Kurz L T, Hecht B, Schwartz M. The effect of interposition membrane on the outcome of lumbar laminectomy and discectomy. Spine (Phila Pa 1976). 1995; 20(16):1793-6.
10. McClinton M A. The use of dermal-fat grafts. Hand Clin. 1996; 12(2):357-64.
11. Dy C J, Aunins B, Brogan D M. Barriers to Epineural Scarring: Role in Treatment of Traumatic Nerve Injury and Chronic Compressive Neuropathy. J Hand Surg Am. 2018:43(4):360-7.
12. Carvalho C R, Oliveira J M, Reis R L. Modern Trends for Peripheral Nerve Repair and Regeneration: Beyond the Hollow Nerve Guidance Conduit. Frontiers in Bioengineering and Biotechnology. 2019; 7.
13. Li X, Cho B, Martin R, Seu M, Zhang C, Zhou Z, et al. Nanofiber-hydrogel composite-mediated angiogenesis for soft tissue reconstruction. Science Translational Medicine. 2019; 11(490):eaau6210.
14. Henn D, Fischer K S, Chen K, Greco A H, Martin R A, Sivaraj D. et al. Enrichment of Nanofiber Hydrogel Composite with Fractionated Fat Promotes Regenerative Macrophage Polarization and Vascularization for Soft-Tissue Engineering. Plast Reconstr Surg. 2022; 149(3):433e-44e.
15. Petersen J, Russell L, Andrus K, MacKinnon M, Silver J, Kliot M. Reduction of extraneural scarring by ADCON-T/N after surgical intervention. Neurosurgery. 1996; 38(5):976-83; discussion 83-4.
16. Dumanian G A, McClinton M A, Brushart T_{M}. The effects of free fat grafts on the stiffness of the rat sciatic nerve and perineural scar. J Hand Surg Am. 1999; 24(1):30-6.
17. Yamamoto M, Endo N, Ito M, Okui N, Koh S, Kaneko H. et al. Novel polysaccharide-derived hydrogel prevents perineural adhesions in a rat model of sciatic nerve adhesion. J Orthop Res. 2010; 28(3):284-8.
18. Wang L X, Zhang S X, Wu H J, Rong X L, Guo J. M2b macrophage polarization and its roles in diseases. J Leukoc Biol. 2019:106(2):345-58.
19. Lopez-Castejon G, Brough D. Understanding the mechanism of IL-10 secretion. Cytokine Growth Factor Rev. 2011; 22(4):189-95.
20. Dinarello C A. Biologic basis for interleukin-1 in disease. Blood. 1996; 87(6):2095-147.
21. Rempel D M, Diao E. Entrapment neuropathies: pathophysiology and pathogenesis. J Electromyogr Kinesiol. 2004; 14(1):71-5.
22. Papanicolaou G D, McCabe S J, Firrell J. The prevalence and characteristics of nerve compression symptoms in the general population. J Hand Surg Am. 2001:26(3):460-6.
23. Fajardo M, Kim S H, Szabo R M. Incidence of carpal tunnel release: trends and implications within the United States ambulatory care setting. J Hand Surg Am. 2012; 37(8):1599-605.
24. Gabrielli A S, Lesiak A C, Fowler J R. The Direct and Indirect Costs to Society of Carpal Tunnel Release. Hand (N Y). 2020; 15(2):Np1-np5.
25. Kanchanathepsak T, Wairojanakul W, Phakdepiboon T, Suppaphol S, Watcharananan I, Tawonsawatruk T. Hypothenar fat pad flap vs conventional open release in primary carpal tunnel syndrome: A randomized controlled trial. World J Orthop. 2017:8(11):846-52.
26. Shamash S, Reichert F, Rotshenker S. The cytokine network of Wallerian degeneration: tumor necrosis factor-alpha, interleukin-1alpha, and interleukin-1beta. J Neurosci. 2002; 22(8):3052-60.
27. Be'eri H, Reichert F, Saada A, Rotshenker S. The cvtokine network of wallerian degeneration: IL-10 and GM-CSF. Eur J Neurosci. 1998; 10(8):2707-13.
28. Kroner A, Greenhalgh A D, Zarruk J G. Passos Dos Santos R, Gaestel M, David S.

TNF and increased intracellular iron alter macrophage polarization to a detrimental M1 phenotype in the injured spinal cord. Neuron. 2014; 83(5):1098-116.
29. Ydens E, Cauwels A, Asselbergh B, Goethals S, Peeraer L, Lomet G, et al. Acute injury in the peripheral nervous system triggers an alternative macrophage response. J Neuroinflammation. 2012; 9:176.
30. O'Kane S, Ferguson M W. Transforming growth factor beta s and wound healing. Int J Biochem Cell Biol. 1997; 29(1):63-78.
31. Steen E H, Wang X, Balaji S. Butte M J, Bollyky P L. Keswani S G. The role of the anti-inflammatory cytokine interleukin-10 in tissue fibrosis. Advances in wound care. 2020; 9(4):184-98.
32. Burd D A R, Ritz M, Regauer S, Longaker M T, Siebert J W, Garg H. Hyaluronan and wound healing: anew perspective. British journal of plastic surgery. 1991; 44(8):579-84.

### Example 4: Collagen Fiber Production

Bovine source type I collagen solution was purchased from Advanced Biomatrix. Bovine collagen solution was firstly lyophilized overnight to obtain collagen powders, and then type I collagen solution (8 w/v %) was prepared in 1,1,1,3,3,3-hexafluoro-2-propanol (HFIP) at room temperature for around 6 hours to make a viscous cloudy electrospinning solution. The electrospinning was performed with the following parameters: 5 mL/h of the flow rate; 20-25 kV of the voltage applied to the 22-G metallic needle; 12.5 cm of the collecting distance: 900 rpm of the rotation rate of the metallic collector. This set of parameters results in a mean fiber diameter of around 600 nm. By using carbodiimide chemistry, fibers were immersed in ethanol solution (95% v/v %) containing 50 mM 1-Ethyl-3-(3-dimethylaminopropyl) carbodiimide (EDC) and 20 mM N-hydroxysuccinimide (NHS) for 24 hours. After the crosslinking, fibers were washed in 0.75% glycine solution three times with 5 minutes each time to remove the excessive reagents and to quench the activated fiber surface. The collagen fibers were then broken down to fragments using cryomilling (Freezer/Mill 6770, SPEX SamplePrep). The fragments were filtered through different cell strainers (40 and 100 μm) to reach a relative uniform fiber length.

### Example 5: Preparation of the Collagen Fiber-HA Hydrogel Composite

A preferred NHC construct comprises composed of three components: hyaluronic acid (HA) network, bovine type I collagen nanofibers, and divinyl sulfone (DVS) crosslinker). Before incorporating the collagen nanofibers (produced in Example 4 above) into the HA network during crosslinking, we optimized the crosslinking conditions for the HA gel phase alone

Sodium hyaluronate (MW 1.5 MDa) was purchased from LifeCore. HUVECs and vascular endothelial cell culture medium were purchased from Lonza. All other chemical reagents were purchased from Sigma-Aldrich. All other cell culture reagents and supplements were obtained from Invitrogen. HA was dissolved in distilled water at a stock concentration of 25 mg/mL. DVS concentration was calculated as the ratio to the hydroxyl groups in HA (such as 1.17 w/v %, 2.34 w/v %, and 4.68 w/v %). The stock HA solution was diluted to 2 w/v % using distilled water and sodium hydroxide to get four different pHs (12.4, 12.7, 13.0 and 13.3), with other parameters set to be the same (2 w/v % HA, 37° C., 3 hour reaction time). The reaction time or gelation kinetics were performed by preparing multiple samples and measuring the mechanical properties at various timepoints (30 min, 1 hour, 2 hours, 3 hours, 4 hours, 8 hours and 16 hours) to get a timepoint where the stiffness reaches a plateau. The crosslinking of the NHCs followed the same conditions to the HA hydrogels, different fiber density (0, 1 and 3 w/v %) were added to the mixed precursors to test the gelation kinetics of the NHCs. After the gelation of hydrogels and NHCs, dialysis was performed using dialysis membranes (6000-8000 MWCO, Spectrum) against pH 7.4 phosphate buffer for 48 hours to remove the unreacted DVS, to balance the pH and to swell the samples for further studies. The mechanical properties were measured again after the swelling. Microgels were then generated with stainless steel wire cloth discs to reach a gel particle size at around 100 μm as we previously reported.

We found that using DVS chemistry to crosslink the HA network at a pH of 12.7 was effective in forming a robust crosslinked hydrogel while minimizing the degradation of HA molecules and collagen fibers during gelation. The reaction pH had a large effect upon the resulting gel in the range of pH 12-13.3. With other parameters set to be the same (37° C., HA concentration 2 w/v %, DVS concentration 2.93 w/v %), while the reaction pH was set to be 12.4, 12.7, 13.0 and 13.3 prepared by different NaOH concentrations (0.001 M, 0.01 M, 0.1 M, 1 M). The reaction at pH 13.3 and 13.0 showed a dramatic degradation after 2 hours of reaction, resulting in two unstable hydrogels with low reproducibility.

The crosslinking time was optimized to around 2 hours to reach the maximal storage modulus and limit degradation. After tuning the DVS chemistry, we introduced the nanofibers to the HA network while crosslinking to generate interfacial bonding between the HA network and the nanofibers, and a reinforcement effect was observed comparing HA and NHC at a similar crosslinking density. Additionally, the composite could pass through 27-gauge needle easily after crosslinking. To further investigate this reinforcement effect quantitatively, we measured the storage modulus G0′ (normalized to a storage modulus control) of the HA hydrogel phase, the G′ of the overall NHC and the G′ of a hydrogel-nanofiber mixture without interfacial bonding. In a rheological test with 1 to 3 w/v % of fiber loading density, the G′ of the composite was ranging from 1.5 to 4 folds higher than that without interfacial bonding, and the G′ difference increased with the increase of the fiber loading and the crosslinker concentration. Furthermore, we also investigated the effect of fiber lengths on the stiffness enhancement by using different cell strainers (40 μm, 100 μm, unscreened) to screen out the large fiber fragments. In an unintuitive result, the gels with the fiber fragments with length between 40 m to 100 m helped generate the largest stiffness enhancement, though this relative enhancement was minimized at the highest crosslinking concentrations. It is not clear why the 40-100 μm fiber size range would be optimal for mechanical reinforcement, as it would generally be expected that longer continuous fibers (>100 μm group) would increase the gel stiffness further by supporting more of the applied load (since the fibrous component is stiffer than the hydrogel phase), or by becoming more entangled (since longer fibers tend to be more prone to entanglement) with other fibers for efficient load transfer (and thus increased stiffness). It was also possible that shorter fibers (<40 μm) would result in a stiffer gel at a given concentration by being less entangled and take on more orientations. It was not expected for the intermediate fibers to result in the most efficient mechanical reinforcement.

In consequence, these tuning steps allowed us to generate NHC with G′ in the range of 450 Pa to 1500 Pa after crosslinking, and 150-Pa to 1000-Pa after swelling. To mimic the soft tissue microenvironment, HA controls (G′=100-Pa and G′=250-Pa) and composite (G′=250-Pa and G0′=100-Pa) were generated and particularized to microgels with diameters around 100 μm. The storage modulus for all three groups were measured not significantly different from the initial crosslinked bulk gels. Lastly, before utilizing the prepared materials for later in vitro and in vivo studies, we autoclaved the hydrogels and composites and observed that the G′ measurements were not significantly lowered after the terminal sterilization, indicating the translational potential of this material. Autoclaving was performed to sterilize the hydrogels and NHCs after gelation. Briefly, the gels were placed at the autoclave cycle at 118° C. with a 5-minute sterilization step. The total sterilization cycle would take 30 minutes to complete. After the sterilization, the mechanical properties of each gel were measured again using rheological tests.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method of treating neural tissue in a subject comprising:
administering to a subject in need thereof a hydrogel composition comprising a polymeric fiber and an associated hydrogel material.

2. A method of treating undesired adhesion formation in a subject comprising:
administering to a subject in need thereof a hydrogel composition comprising a polymeric fiber and an associated hydrogel material.

3. A method of treating a subject suffering from or susceptible to carpal tunnel syndrome, radiation-induced fibrosis or arthrofibrosis, comprising:
administering to the subject in need thereof a hydrogel composition comprising a polymeric fiber and an associated hydrogel material.

4-5. (canceled)

6. The method of claim 1, wherein the neural tissue comprises peripheral nerves.

7. The method of claim 1, wherein the subject is treated with neurosurgery.

8. The method of claim 1, wherein the subject is treated with nerve decompression and/or neurolysis.

9. The method of claim 1 wherein the hydrogel composition is capable of at least one of i) suppressing scarring, ii) suppressing adhesion of the neural tissues, and/or iii) promoting regeneration of the neuton tissued in the subject.

10. The method of claim 1 wherein the hydrogel composition is injected or implanted on or around neural tissue of the subject.

11. The method of claim 1 wherein the hydrogel composition is administered dermally or subdermally to treat neural tissue of the subject.

12. The method of claim 1 wherein the polymeric fiber is covalently linked to hydrogel material.

13. The method of claim 1 wherein the hydrogel composition is applied to or around the neural tissues.

14. The method of claim 1 wherein the hydrogel composition comprises a functionalized hyaluronic acid network covalently linked to a plurality of fibers.

15. The method of claim 1 wherein the hydrogel composition comprise a functionalized hyaluronic acid.

16. The method of claim 15 wherein the functionalized hyaluronic acid comprises thiolated hyaluronic acid, and the crosslinking agent comprises poly(ethylene glycol) diacrylate (PEGDA).

17. The method of claim 1 wherein the polymeric fiber comprises a plurality of polycaprolactone fibers.

18. The method of claim 1 wherein the hydrogel composition comprise a collagen material.

19. (canceled)

20. The method of claim 1 wherein the hydrogel composite further comprises one or more compounds that have therapeutic effects, vascularization effects, anti-vascularization effects, anti-inflammatory effects, anti-bacterial effects, antihistamine effects, and combinations thereof.

21. The method of claim 1 wherein the subject is not in need of a dermal filler in the area the hydrogel composition is administered.

22. The method of claim 1 wherein the subject is not in need of treatment for wound healing.

23. A implant for treating a subject after neurosurgery, comprising a hydrogel composite comprising a functionalized hyaluronic acid network covalently linked to a plurality of nanofibers, wherein the implant is configured to be applied on or around neural tissues of the subject.

24-33. (canceled)

34. A kit comprising the implant of claim 23.

35-42.
